(19) United States
(12) Patent Application Publication
(10) Pub. No.: US 2025/0195917 A1
(43) Pub. Date: Jun. 19, 2025

(54) SEQUENTIAL TRANSARTERIAL CHEMOEMBOLIZATION, STEREOTACTIC BODY RADIOTHERAPY, AND IMMUNOTHERAPY (START-FIT) FOR HEPATOCELLULAR CARCINOMA

(71) Applicant: The University of Hong Kong, Hong Kong (CN)

(72) Inventors: Chi Yan Albert Chan, Hong Kong (CN); Chi Leung Chiang, Hong Kong (CN)

(21) Appl. No.: 18/538,932

(22) Filed: Dec. 13, 2023

(51) Int. Cl. A61N 5/10; A61N 1/40; A61P 35/00; C07K 16/28

(52) U.S. Cl. CPC A61N 5/1001; A61N 1/40; A61P 35/00; C07K 16/2818

(57) ABSTRACT

Disclosed are methods and materials for treating a subject having hepatocellular carcinoma (HCC). The methods and materials are useful, for example, for conversion of locally advanced, unresectable HCC. For example, the disclosed methods and materials are useful for conversion of locally advanced, unresectable HCC. The methods generally include sequential combination of three treatments: transarterial chemoembolization (TACE); stereotactic body radiotherapy (SBRT); and immunotherapy with an immune checkpoint inhibitor.

### FIELD OF THE INVENTION

The disclosed invention is generally in the field of cancer treatment and specifically in the area of treatment of hepatocellular carcinoma.

### BACKGROUND OF THE INVENTION

Hepatocellular carcinoma is the sixth most common cancer globally and the third leading cause of cancer-related mortality.¹ Transarterial chemoembolisation (TACE) is the most widely adopted therapy for patients with unresectable, liver-limited hepatocellular carcinoma. However, TACE was associated with low response rates (roughly 30%) in patients with large (>5 cm) or multinodular hepatocellular carcinoma.^{2,3} In the presence of vascular invasion, systemic therapy is the standard of care, yet the response rates were modest, ranging between 5% and 40%.⁴⁻⁶

There is a need for a conversion therapy for locally advanced, unresectable hepatocellular carcinoma (HCC).

It is therefore an objection of the present invention to provide a method that improves conversion of HCC over conventional therapies for HCC.

### BRIEF SUMMARY OF THE INVENTION

It has been discovered that a sequential combination of three different treatments produces a treatment regime that produces downstaging conversion of locally advanced, unresectable HCC. A variety of treatments have been tried on locally advanced, unresectable HCC. Prior to the present disclosure, no conversion treatment regimen for locally advanced, unresectable HCC has been established.

Disclosed are methods and materials for treating a subject having hepatocellular carcinoma (HCC). The methods and materials are useful, for example, for conversion of locally advanced, unresectable HCC. For example, the disclosed methods and materials are useful for conversion of locally advanced, unresectable HCC. The methods generally include sequential combination of three treatments: transarterial chemoembolization (TACE); stereotactic body radiotherapy (SBRT); and immunotherapy with an immune checkpoint inhibitor.

In some forms, the subject's HCC is locally advanced. In some forms, the subject's HCC is unresectable. In some forms, the subject's HCC is locally advanced and unresectable.

In some forms, the treatments are performed sequentially in order of transarterial chemoembolization (TACE); stereotactic body radiotherapy (SBRT); and immunotherapy with an immune checkpoint inhibitor.

In some forms, the immune checkpoint inhibitor comprises a therapeutic antibody.

In some forms, the immune checkpoint inhibitor targets programmed cell death ligand 1 (PD-L1). In some forms, the immune checkpoint inhibitor comprises a therapeutic antibody, and the therapeutic antibody is avelumab, atezolizumab, durvalumab, envafolimab, cosibelimab, or AUNP-12.

In some forms, the immune checkpoint inhibitor targets programmed cell death protein 1 (PD-1). In some forms, the immune checkpoint inhibitor comprises a therapeutic antibody, and the therapeutic antibody is pembrolizumab, nivolumab, cemiplimab, spartalizumab, camrelizumab, sintilimab, tislelizumab, toripalimab, dostarlimab, retifanlimab, AMP-224, or MEDI0680.

In some forms, the immune checkpoint inhibitor targets cytotoxic T-lymphocyte-associated protein 4 (CTLA-4). In some forms, the immune checkpoint inhibitor comprises a therapeutic antibody, and the therapeutic antibody is ipilimumab or tremelimumab.

In some forms, the immune checkpoint inhibitor targets lymphocyte-activation gene 3 (LAG-3). In some forms, the immune checkpoint inhibitor comprises a therapeutic antibody, and the therapeutic antibody is relatlimab.

In some forms, TACE comprises infusing a chemotherapeutic agent and an embolic particle into an artery supplying blood to one or more tumors of the HCC. In some forms, the chemotherapeutic agent and the embolic particle are infused together or separately. In some forms, the chemotherapeutic agent and the embolic particle are infused sequentially. In some forms, the embolic particle comprises a polyvinyl alcohol microsphere, superabsorbent microsphere, gelatin microsphere, or degradable starch microsphere.

In some forms, the method also includes resecting tumor tissue in the subject. In some forms, the method also includes treating the subject with radiofrequency ablation.

### BRIEF DESCRIPTION OF THE DRAWINGS

FIG. 1 is a schematic of trial profile. *29 patients were excluded for the following reasons: more than three lesions (n=11); extrahepatic vascular invasion (main portal vein thrombosis [Vp4] or inferior vena cava [VV3] involvement; n=5); extrahepatic metastases (n=4); poor liver function (n=2); liver volume minus gross tumor volume less than 700 mL (n=3); secondary malignancy (n=2); poor renal function (n=2). †One patient did not receive avelumab due to disease progression.

FIGS. 2A-2C are plots showing tumor responses. FIG. 2A is a waterfall plot showing the percentage change from baseline in the sum of the longest diameter of target lesions in each of the 33 patients, according to treatment response per modified RECIST. FIG. 2B is a swimmer plot showing the duration of response and time to response. Bars signify duration of response. FIG. 2C is a spider plot showing the percentage change from baseline in the sum of longest diameter of target lesions over time (months) in each of the 33 patients, according to treatment response per modified RECIST. RECIST=Response Evaluation Criteria in Solid Tumors. Dotted lines in FIG. 2A and FIG. 2C represent the definition of partial response and progressive disease per modified RECIST criteria. *Patients with a new lesion or lesions or progression of a non-target lesion. †Treated with curative intent.

FIGS. 3A-3C are plots showing survival outcomes and local control. Progression-free survival, per patient (n=33) (FIG. 3A). Overall survival, per patient (n=33) (FIG. 3B). Local control, per lesion (n=55) (FIG. 3C). NR=not reached.

FIGS. 4A-4C are MRIs of examples of patients treated with sequential TACE and SBRT followed by immunotherapy. FIG. 4A shows scans of BCLC stage C1 without extra-hepatic metastases (Patient #5): A 75-year-old lady with (1, baseline scan) HCC (11.6 cm) at segment 4/8 with left, right, and hepatic vein invasion, treated with TACE followed by SBRT (27.5 Gy in 5 fractions) and avelumab every 2 week for 5 doses. (2, post-treatment scan) MRI showed complete remission (mRECIST) and then patient was put on surveillance. The disease remains in remission after follow-up for 26 months. FIG. 4B shows scans of BCLC stage B and up-to-seven out (Patient #15): (1, baseline scan) 59-year-old male had two HCCs (10.2 cm, 7.2 cm) was successfully downstaged after TACE and SBRT followed by avelumab. (2, post-treatment scan) MRI showed significant decrease in size of tumors (3.6 cm, 2.3 cm). Trisectionectomy was subsequently performed and pathology showed 60% necrosis of tumor. FIG. 4C shows scans of inadequate liver volume (Patient #10): (1, baseline scan) A 69-year-old male with HBV related liver cirrhosis, Child-Pugh B7 liver function, was diagnosed to have 5.3 cm HCC. TACE followed by SBRT (30 Gy in 5 fractions) followed by 10 doses of avelumab. (2, post-treatment with 1 cm residual) MRI showed 1 cm residual tumor, which was ablated by radiofrequency ablation. (3, post-RFA with complete remission) The disease was in complete remission after 22 months of follow-up.

FIGS. 5A-5B are plots of overall survival versus time of OS (FIG. 5A) and PFS (FIG. 5B) in patients without macrovascular invasion (n=12) versus those with macrovascular invasion (n=21).

FIGS. 6A-6K are graphs showing mean quality of life (QoL) scores with 95% confidence interval for Quality of Life Questionnaire Core-30 domains (EORTC QLQ-C30). For functional scales, higher score indicates better QoL. For symptom scales, lower score indicates better QoL.

FIGS. 7A-7J are graphs showing mean quality of life score with 95% confidence interval for Functional Assessment of Cancer Therapy-Hepatobiliary (FACT-Hep) domains. Higher score indicates better quality of life.

FIG. 8 is a plot showing median serum AFP with time. (*)P<0.05 for difference from baseline using Wilcoxon signed-rank test. Thin vertical bars are interquartile ranges.

FIGS. 9A-9B are plots of overall survival versus time of OS (FIG. 9A) and PFS (FIG. 9B) in patients with Grade 1 ALBI (n=21) vs. Grade 2 ALBI (n=12).

FIGS. 10A-10B are graphs of complete response rate of subjects with low (<250 μg/ml) and high (≥250 μg/ml) of exosomal PD-L1 level among complete responders vs. non-complete responders at baseline and at 1 month after immunotherapy.

### DETAILED DESCRIPTION OF THE INVENTION

The disclosed method and compositions can be understood more readily by reference to the following detailed description of particular embodiments and the Example included therein and to the Figures and their previous and following description.

It has been discovered that a sequential combination of three different treatments produces a treatment regime that produces downstaging conversion of locally advanced, unresectable HCC. A variety of treatments have been tried on locally advanced, unresectable HCC. Neither TACE nor systemic therapy has been effective to downstage locally advanced, unresectable hepatocellular carcinoma for curative surgery. Stereotactic body radiotherapy has produced some tumor response in patients with locally advanced hepatocellular carcinoma.⁷ And local response rates were enhanced when combined with TACE.^{8,9} However, prior to the present work, no conversion treatment regimen for locally advanced, unresectable HCC has been established.

Although the combination of stereotactic body radiotherapy and a single course of TACE provided some improvement in antitumor response and survival benefits compared to repeated TACE,¹⁰ out-of-field disease progression remains a substantial drawback to locoregional therapy. A thought occurred to combine systemic therapy with TACE and stereotactic body radiotherapy. It was realized that locoregional therapy could prime the immune system and modulate the tumor microenvironment, with a resulting enhancement in the response to immune checkpoint inhibitors. It was discovered that such an enhanced immune response following locoregional therapy could be harnessed to improve the effect of TACE and stereotactic body radiotherapy.

The present triple combination of sequential TACE, stereotactic body radiotherapy, and immune checkpoint inhibitor was discovered. This sequential triple therapy, dubbed START-FIT, can provide downstaging conversion in patients with locally advanced unresectable hepatocellular carcinoma.

It is to be understood that the disclosed method and compositions are not limited to specific methods, specific analytical techniques, or to particular materials or reagents unless otherwise specified, and, as such, can vary. It is also to be understood that the terminology used herein is for the purpose of describing particular embodiments only and is not intended to be limiting.

### Methods

Disclosed are methods and materials for treating a subject having hepatocellular carcinoma (HCC). The methods and materials are useful, for example, for conversion of locally advanced, unresectable HCC. For example, the disclosed methods and materials are useful for conversion of locally advanced, unresectable HCC. In some forms, the subject's HCC is locally advanced. In some forms, the subject's HCC is unresectable. In some forms, In some forms, the subject's HCC is locally advanced and unresectable.

### A. Hepatocellular Carcinoma (HCC)

Hepatocellular carcinoma (HCC) is the most common type of primary liver cancer. It originates in the hepatocytes, which are the main functional cells of the liver. HCC typically occurs in the context of underlying liver disease, such as cirrhosis or chronic hepatitis infection, and it is often associated with long-term alcohol abuse or viral hepatitis infection, particularly hepatitis B or hepatitis C. Here are some key points about hepatocellular carcinoma:

Risk Factors: It isn't exactly established what causes all cases of hepatocellular carcinoma, but several risk factors have been identified that increase the likelihood of developing HCC. These include chronic liver diseases like cirrhosis, chronic viral hepatitis (especially hepatitis B and C), excessive alcohol consumption, non-alcoholic fatty liver disease, obesity and diabetes, anabolic steroids, iron storage disease (that cause too much iron to be stored in the liver) and exposure to certain toxins or carcinogens.

Symptoms: In its early stages, HCC may not cause noticeable symptoms. As it progresses, common symptoms may include abdominal pain or discomfort, jaundice (yellowing of the skin and eyes), unexplained weight loss, fatigue, nausea and vomiting and a feeling of fullness in the abdomen.

Diagnosis: HCC is typically diagnosed through imaging tests like ultrasound, CT scans, or MRI scans, as well as blood tests that measure liver function and tumor markers. A biopsy may also be performed to confirm the diagnosis.

Staging: Staging helps determine the extent of the cancer and guides treatment decisions. The Barcelona Clinic Liver Cancer (BCLC) staging system is often used for HCC. It takes into account factors such as tumor size, number of tumors, liver function, and overall health.

Prevention: Preventing HCC is closely linked to managing risk factors. Vaccination against hepatitis B, screening and early treatment for hepatitis B and C, limiting alcohol consumption, and managing conditions like cirrhosis and NAFLD can all reduce the risk of developing HCC.

### 1. Locally Advanced HCC

“Locally advanced” hepatocellular carcinoma (HCC) is a term used to describe tumor that has progressed beyond its initial site of origin but has not yet spread extensively to distant parts of the body. This term encompasses a heterogeneous morphology with no regard to stage of prognosis of the disease. In other words, locally advanced cancer has grown into nearby tissues, organs, or structures, but it has not metastasized, which means it has not spread to distant organs or distant lymph nodes.

The exact definition of “locally advanced” can also depend on the stage of cancer, but it generally means that the tumor has reached a stage where it is often challenging to treat with localized therapies like surgery or radiation therapy alone. Treatment for locally advanced HCC can combine chemotherapy, embolization, chemoembolization (such as TACE), radiation therapy (including SBRT), immunotherapy (such as immune checkpoint inhibitors), or any combination of these in any sequence or manner.

### 2. Unresectable HCC

“Unresectable” HCC can be defined as a liver tumor or cancerous growth that cannot be completely removed or excised given the extent of disease, including patients that are not suitable for surgery for location of tumor(s) in the liver, or patients who were older than 75 years, or those who refused surgical therapies. This also means that if surgically removed, it can cause excessive damage to vital organs, tissues, or structures.

The unresectability of HCC can be attributed to several reasons, including tumor location (some tumors can be located in areas that are difficult to access surgically, such as deep within vital organs or near critical blood vessels or nerves), tumor size (extremely large tumors may be technically impossible to remove in their entirety, especially if their size makes surgical removal too risky), metastasis (when cancer has spread to distant organs or has metastasized extensively throughout the body, it may no longer be feasible to surgically remove all affected areas), or poor general health (patients with significant comorbidities or underlying medical conditions may not be suitable candidates for extensive surgical procedures).

Treatment options for Hepatocellular carcinoma HCC depend on the stage of the cancer, the patient's overall health, and other factors. Treatment may include surgical options like resection or liver transplantation, local therapies transarterial chemoembolization (TACE) or stereotactic body radiotherapy (SBRT), systemic therapies like targeted therapies or immunotherapy, and supportive care to manage symptoms and improve quality of life. Additionally, another option includes combination of therapies that can incorporate treatment with TACE, SBRT, immunotherapy, in any combination or manner.

### B. Sequential Triple Treatment

The methods generally include sequential combination of three treatments: transarterial chemoembolization (TACE); stereotactic body radiotherapy (SBRT); and immunotherapy with an immune checkpoint inhibitor. In some forms, the treatments are performed sequentially in order of transarterial chemoembolization (TACE); stereotactic body radiotherapy (SBRT); and immunotherapy with an immune checkpoint inhibitor.

### 1. Transarterial Chemoembolization (TACE)

TACE generally includes infusing a chemotherapeutic agent and an embolic particle into an artery supplying blood to one or more tumors of the HCC. In some forms, the chemotherapeutic agent and the embolic particle are infused together or separately. In some forms, the chemotherapeutic agent and the embolic particle are infused sequentially. In some forms, the embolic particle comprises a polyvinyl alcohol microsphere, superabsorbent microsphere, gelatin microsphere, or degradable starch microsphere.

TACE represents a minimally-invasive, image-guided approach to addressing liver cancer. This technique aims to reduce or eliminate tumors by targeting them, obstructing their blood supply, and administering chemotherapy directly to the tumor site. TACE enables medical professionals to manage tumors that cannot be reached through traditional surgical or radiation methods. TACE is primarily used to treat liver cancer.

TACE generally involves patient evaluation and preparation, arterial catheterization, chemotherapy, embolization, monitoring and imaging, and completion and recovery.

i. Patient Evaluation and Preparation

Before TACE, a patient's eligibility for the procedure is carefully assessed. This evaluation includes reviewing the patient's medical history, conducting physical examinations, and assessing liver function and overall health. Imaging studies, such as CT scans, MRI scans, or angiography, are often used to precisely locate and characterize the liver tumor(s). The patient may receive mild sedation or local anesthesia to minimize discomfort during the procedure. An intravenous (IV) line is typically placed in the patient's arm to administer medications and fluids during the procedure.

ii. Arterial Catheterization

The procedure begins with the insertion of a thin, flexible tube called a catheter into a large artery, often in the groin (femoral artery). This is done using a small incision. The catheter is guided through the arterial system using real-time imaging guidance, such as fluoroscopy or angiography.

The goal is to advance the catheter into the hepatic artery, which supplies blood to the liver and the tumor.

iii. Chemotherapy

During TACE, a high dose of chemotherapy drugs is delivered directly into the arteries that supply blood to the tumor in the liver. This allows for a concentrated and targeted delivery of chemotherapy to the cancerous tissue while minimizing the systemic side effects of the drugs on the rest of the body.

Chemotherapy is a common treatment method for cancer that uses drugs to destroy or slow down the growth of cancer cells. There are several methods for administering chemotherapy, and the choice of method depends on the type of cancer, its stage, and the patient's overall health. Here are some of the methods of treating cancer with chemotherapy.

Intravenous (IV) Chemotherapy. This is one of the most common methods of administering chemotherapy. The drugs are injected directly into a vein, allowing them to quickly enter the bloodstream and circulate throughout the body. IV chemotherapy can be given through a catheter, port, or by direct injection into a vein.

Oral Chemotherapy. Some chemotherapy drugs are available in pill or liquid form, which can be taken orally. Patients can take these medications at home as prescribed by their oncologist.

Intramuscular (IM) or Subcutaneous (SC) Injections. In some cases, chemotherapy drugs are administered as injections into the muscle (IM) or just under the skin (SC).

Intra-arterial Chemotherapy. This method delivers chemotherapy drugs directly into the artery that supplies blood to the tumor. It is often used in cases where a specific area of the body needs targeted treatment, such as liver cancer or some types of sarcoma.

Intraperitoneal Chemotherapy. Chemotherapy drugs can be delivered directly into the abdominal cavity for the treatment of cancers that have spread to this area, such as ovarian cancer or peritoneal mesothelioma.

Regional Chemotherapy. In this approach, the chemotherapy drugs are delivered directly to a specific region of the body where the cancer is located, often using a catheter or specialized devices.

Continuous Infusion. Some chemotherapy drugs are administered continuously over an extended period through a small portable pump. This method allows for a controlled and steady release of the drugs.

There are several classes of, and numerous specific chemotherapy agents that can be used to treat cancer. Examples are described in Table 1.

TABLE 1
Examples of Classes of and Specific Chemotherapeutics

| Classes of Chemotherapeutics | Specific Chemotherapeutics |
|---|---|
| Alkylating agents: | Altretamine, Busulfan, Carboplatin, |
| These work by alkylating (adding alkyl groups) to DNA | Carmustine, Cisplatin, |
| molecules which interfere with cancer cell's growth and | Cyclophosphamide, Dacarbazine, |
| division. These drugs also disrupt DNA structure. Sone | Ifosfamide, Lomustine, Melphalan, |
| of the drug cause DNA cross-linking and damage. | Temozolomide, Trabectedi |
| Antimetabolites: | 5-Fluorouracil, 6-Mercaptopurine, |
| These drugs work by mimicking metabolites within cells. | Azacitidine, Capecitabine, |
| These metabolites disrupt DNA and RNA synthesis thus | Clofarabine, Cytarabine, |
| interfering with the cells ability to replicate and divide. | Floxuridine, Fludarabine, |
| | Gemcitabine, Methotrexate, |
| | Pemetrexed, Pentostatin, |
| | Pralatrexate, Trifluridine, Tipiracil |
| Plant alkaloids: | Vincristine, Vinblastine, |
| These are plant-based drugs that prevent cells from | Vinorelbine, Paclitaxel, Docetaxel, |
| growing and dividing. Some of these function as | Etoposide, Teniposide, Irinotecan, |
| topoisomerase inhibitors or microtubule inhibitors. | Topotecan |
| Antitumor Antibiotics: | Bleomycin, Daunorubicin, |
| These drugs work by causing DNA strands to unravel | Doxorubicin, Liposomal, |
| or inhibiting DNA synthesis by binding to DNA and | Doxorubicin, Epirubicin, |
| preventing its proper replication and repair. | Idarubicin, Valrubicin |
| Hormone modulators: | Tamoxifen, Anastrozole, Letrozole |
| These are used to treat hormone receptor-positive | |
| cancers. They interfere with hormone signaling | |
| pathways, either by blocking hormone receptors or | |
| inhibiting hormone production. | |
| Targeted Therapies: | Imatinib, Trastuzumab, Rituximab, |
| These drugs specifically target proteins or pathways | Erlotinib, Lapatinib, Sorafenib, |
| involved in cancer cell growth and survival, and work | Sunitinib |
| by inhibiting or blocking these specific targets. | |

iv. Embolization

Embolization involves blocking or reducing the blood supply to the tumor with the goal of cutting off its supply of oxygen and nutrients to inhibit tumor growth or shrink the tumor. In TACE, small particles or beads are often used to block the arteries feeding the tumor. This cut-off of blood flow to the tumor not only reduces the tumor's access to nutrients but also helps trap the chemotherapy drugs within the tumor, enhancing their effectiveness. The choice of embolic material (e.g., beads, particles) may vary based on the specific tumor and patient characteristics. Here are the examples of embolizing particles used in cancer treatments.

Microspheres. Microspheres are small, spherical particles made of various materials that can be used to block blood vessels. Examples are Polyvinyl alcohol (PVA) microspheres and Embospheres. Embospheres are made from a biocompatible material.

Gelatin Sponge Particles. Gelatin sponge particles are absorbable and can be used to create temporary occlusion of blood vessels. One example is Gelfoam which is a biocompatible gelatin sponge used for a variety of embolization procedures.

Coils. Coils are metal devices that are inserted into blood vessels to mechanically block blood flow. Examples include detachable coils such as Nester coils and Concerto coils.

Polymeric Particles. Polymeric particles are often used for targeted embolization procedures, such as those involving the brain or other sensitive areas. Examples include (1) precipitating hydrophobic injectable liquid (PHIL) which is a polymer-based embolic agent used in neurovascular embolization, (2) Onyx which is a non-adhesive liquid embolic agent used in neurovascular embolization. (Faragò, et al., The Neuroradiology Journal 26, 678-682 (2013).)

Spheres Loaded with Chemotherapy. Some embolic particles are designed to carry chemotherapy drugs directly to the tumor site during embolization. This is known as drug-eluting embolization. Examples include DC Bead which can be loaded with chemotherapy drugs like doxorubicin for targeted treatment in procedures like TACE (Transarterial Chemoembolization).

Radioactive Particles. Radioactive particles can be used in certain cancer treatments to deliver targeted radiation therapy (radioembolization). Examples include Yttrium-90 microspheres (SIR-Spheres) which can be used in radioembolization cancer treatment. (Khajornjiraphan, et al., Liver Cancer 4, 6-15 (2014)).

Starch Microspheres. Starch microspheres are used in some embolization procedures, particularly for treating vascular malformations. Examples include EmboGold microspheres.

v. Monitoring and Imaging

Throughout the procedure, the interventional radiologist continuously monitors the progress using imaging techniques like fluoroscopy or angiography. These images help ensure that the chemotherapy drugs are being distributed within the tumor and that embolization is effectively reducing blood flow to the tumor. Adjustments may be made during the procedure to optimize the treatment.

vi. Completion and Recovery

Once the treatment is deemed complete, the catheter is carefully removed. Pressure is applied to the site where the catheter was inserted to prevent bleeding. The patient is observed for a period to monitor vital signs and ensure there are no immediate complications. Depending on the patient's condition, they may be discharged on the same day or kept for overnight observation.

### 2. Stereotactic Body Radiotherapy (SBRT)

Stereotactic body radiotherapy (SBRT) is a type of radiation therapy that uses highly precise and targeted form of radiation to treat cancer. There are several types or techniques within the broader category of SBRT, each with its unique approach to delivering high-dose radiation to tumors while minimizing exposure to surrounding healthy tissues. Described below are some common types of SBRT.

Cone-Beam CT-Based SBRT. This technique uses cone-beam computed tomography (CBCT) for image-guidance. It involves taking frequent CT scans during treatment sessions to verify the tumor's position and adjust the radiation beams as needed.

CyberKnife. CyberKnife is a specialized robotic radiosurgery system that delivers high-dose radiation with extreme accuracy. It uses real-time image guidance and tracking to adapt to the patient's breathing and movement during treatment, making it particularly suitable for tumors in the lung, liver, and other mobile organs. The CyberKnife System is a non-invasive treatment for cancerous and non-cancerous tumors and other conditions where radiation therapy is indicated. It is used to treat conditions throughout the body, including the prostate, lung, brain, spine, head and neck, liver, pancreas and kidney, and can be an alternative to surgery or for patients who have inoperable or surgically complex tumors. CyberKnife treatments are typically performed in 1 to 5 sessions.

TrueBeam STx. TrueBeam STx is a linear accelerator (LINAC)-based SBRT system that allows medical doctors to target tumors. The system uses superior imaging technology to capture images of tumors, even when they move during patients natural breathing patterns. It uses these images to confirm that the radiation beams are always targeting tumors. And, because tumors aren't perfectly round, TrueBeam STx can alter the shape of the radiation beam to match the shape of tumors. This may decrease the amount of radiation to healthy tissue that surrounds the tumor.

RapidArc SBRT. RapidArc radiation therapy is a technique that combines intensity modulated radiation therapy (IMRT) with rotational arc delivery on the linear accelerator. The treatment consists of 360-degree rotation of the accelerators gantry with the beam focused on the target. This dynamic use of software and hardware makes for the most sophisticated treatment delivery technologies. During treatment, the radiation beams are consistently shaped to conform to the shape of the tumor as the linear accelerator rotates around the patient. Each treatment is typically completed in less than two minutes. Treatment speed is important because it reduces the amount of time that the patient must remain still and avoid movement.

Respiratory Gating. Gating is a system that tracks a patient's normal respiratory cycle with an infrared camera and chest/abdomen marker. The system is coordinated to only deliver radiation when the tumor is in the treatment field. It is often employed in SBRT to account for tumor motion during the breathing cycle, allowing precise radiation delivery during specific phases of respiration.

Frameless SBRT. Frameless SBRT refers to SBRT procedures that do not require the use of a stereotactic frame to immobilize the patient. Instead, advanced immobilization devices and image guidance are used to ensure accurate tumor targeting.

Gamma Knife Perfexion. Gamma Knife Perfexion is a specialized system for delivering stereotactic radiosurgery (SRS) to treat brain tumors and conditions. Gamma Knife radiosurgery is one of the most advanced and non-invasive options for brain-related conditions like, malignant and benign brain tumors, arteriovenous malformations, tremor due to essential tremor or Parkinson's disease and trigeminal neuralgia. It uses precisely focused beams of gamma radiation to target tumors.

Proton Beam SBRT. Proton therapy delivers radiation using proton beams, which have unique characteristics that allow for precise tumor targeting and minimal damage to surrounding healthy tissues. Proton beam SBRT is used for specific tumor types, including certain pediatric cancers and tumors located near critical structures. With the precision of proton pencil beam scanning (PBS), SBRT to all sites can be performed using protons.

SBRT is characterized by patient immobilization, limiting normal tissue exposure to high-dose radiation, preventing or accounting for organ motion, the use of stereotaxy, and the sub-centimeter accuracy of the delivered dose. The key components of a SBRT procedure are target delineation, treatment planning, and treatment delivery. The treatment team includes a radiation oncologist, medical physicist, radiation therapist, and depending on the body site and indication, a diagnostic radiologist, surgeon, nurse, and dosimetrist as needed.

### 3. Immune Checkpoint Inhibitor Immunotherapy

Immunotherapy is a type of medical treatment that harnesses the body's own immune system to combat diseases, particularly cancer and certain autoimmune disorders. The goal of immunotherapy is to stimulate or enhance the body's natural defenses against harmful cells or substances, such as cancer cells or pathogens. There are different approaches to immunotherapy, but one of the common forms of immunotherapy include immune checkpoint inhibitors.

Immune checkpoints refer to inhibitory pathways hardwired into the immune system that are crucial for maintaining self-tolerance and modulating the duration and amplitude of physiological immune responses in peripheral tissues in order to minimize collateral tissue damage. Immune checkpoint molecules can be stimulatory or inhibitory to an immune checkpoint. The present disclosure and claims refer to inhibitory molecules of immune checkpoints as “immune checkpoint molecules.”

Agents that block immune checkpoint molecules (e.g. PD-1, PD-L1, CTLA-4, or LAG-3), suggest opportunities to enhance antitumor immunity with the potential to produce effective clinical responses. The present application discloses that combining immune checkpoint blockade using immune checkpoint inhibitor(s) with TACE and SBRT enhances treatment efficacy in a subject having HCC.

An immune checkpoint inhibitor is a type of drug that blocks the signaling of immune checkpoint molecule(s) made by some types of immune system cells, such as T cells and some cancer cells. Immune checkpoint inhibitors therefore can cause immune checkpoint blockade. Immune checkpoint molecules (e.g., PD-1) help keep immune responses in check and can keep T cells from killing cancer cells. When these molecules are blocked, the “brakes” on the immune system are released (inhibition of the immune system is reduced or blocked) and T cells are able to kill cancer cells better. Examples of checkpoint proteins found on T cells or cancer cells include PD-1, PD-L1, CTLA-4, and LAG-3. In some embodiments, immune checkpoint molecules are proteins. In some embodiments, immune checkpoint molecules are nucleic acids that encode the proteins. In some embodiments, immune checkpoint inhibitors bind to and/or antagonize immune checkpoint molecules.

The agent that binds to and/or antagonizes an immune checkpoint molecule is an immune checkpoint inhibitor. One or more immune checkpoint inhibitors refer to one or more different inhibitors. Each different inhibitor has a different molecular structure. Two different inhibitors may bind the same immune checkpoint molecule, or each may bind a different immune checkpoint molecule.

An inhibitor or antagonist, as used herein, is a molecule that inhibits, reduces, or blocks activity of an immune checkpoint molecule to inhibit a suppressive effect that the immune checkpoint molecule has on the immune system. The inhibitor or antagonist can directly bind the immune checkpoint molecule, a molecule controlling the expression of the immune checkpoint molecule, or a ligand of the immune checkpoint molecule that mediates the activity of the immune checkpoint molecule. The inhibitor or antagonist may be an antibody (including a humanized antibody), a small molecule, a peptide, or a nucleic acid (e.g., an antisense molecule, or a single- or double-stranded RNAi molecule). Activity of the immune checkpoint molecule is referred to as its suppressive effect on an immune checkpoint. An immune checkpoint inhibitor can reduce or block the activity of an immune checkpoint molecule.

In some forms, the immune checkpoint inhibitor comprises a therapeutic antibody. In some forms, the immune checkpoint inhibitor targets programmed cell death ligand 1 (PD-L1). In some forms, the immune checkpoint inhibitor comprises a therapeutic antibody, and the therapeutic antibody is avelumab, atezolizumab, durvalumab, envafolimab, cosibelimab, or AUNP-12.

In some forms, the immune checkpoint inhibitor targets programmed cell death protein 1 (PD-1). In some forms, the immune checkpoint inhibitor comprises a therapeutic antibody, and the therapeutic antibody is pembrolizumab, nivolumab, cemiplimab, spartalizumab, camrelizumab, sintilimab, tislelizumab, toripalimab, dostarlimab, retifanlimab, AMP-224, or MEDI0680.

In some forms, the immune checkpoint inhibitor targets cytotoxic T-lymphocyte-associated protein 4 (CTLA-4). In some forms, the immune checkpoint inhibitor comprises a therapeutic antibody, and the therapeutic antibody is ipilimumab or tremelimumab.

In some forms, the immune checkpoint inhibitor targets lymphocyte-activation gene 3 (LAG-3). In some forms, the immune checkpoint inhibitor comprises a therapeutic antibody, and the therapeutic antibody is relatlimab.

i. Immunotherapy Targets
a. Programmed Cell Death Ligand 1 (PD-L1)

In humans, programmed death-ligand 1 (PD-L1), also known as B7 homolog 1 (B7-H1) or cluster of differentiation 274 (CD274), is a 40 kDa type 1 transmembrane protein that is encoded by the CD274 gene. Foreign antigens normally induce an immune response triggering proliferation of antigen-specific T cells, such as antigen-specific CD8+ T cells. PD-L1 is an immune checkpoint inhibitor that may block or lower such an immune response. PD-L1 may play a major role in suppressing the immune system during events such as pregnancy, tissue allografts, autoimmune disease, and other disease states, such as hepatitis and cancer. The PD-L1 ligand binds to its receptor, PD-1, found on activated T cells, B cells, and myeloid cells, thereby modulating activation or inhibition. In addition to PD-1, PD-L1 also has an affinity for the costimulatory molecule CD80 (B7-1).

PD-L1 Antagonist. A PD-L1 antagonist, as used herein, is a molecule that binds to PD-L1 protein or to a gene or nucleic acid encoding PD-L1 protein and inhibits or prevents PD-1 activation. Without wishing to be bound by theory, it is believed that such molecules reduce or block the interaction of PD-L1 with PD-1. PD-L1 activity may be blocked by molecules that selectively bind to and block the activity of PD-L1. Anti-PD-L1 antibodies block interactions between PD-L1 and both PD-1 and B7-1 (also known as CD80).

Block means inhibit or prevent the transmission of an inhibitory signal mediated via such PD-L1 binding. PD-L1 antagonists include, for example: BMS-936559, also known as MDX-1105 (Bristol-Meyers Squibb), a fully human, high affinity, immunoglobulin (Ig) G4 monoclonal antibody to PD-L; MPDL3280A, also known as RG7446 or atezolizumab (Genentech/Roche), an engineered human monoclonal antibody targeting PD-L1; MSB0010718C, also known as avelumab (Merck), a fully human IgG1 monoclonal antibody that binds to PD-L1; and MEDI473 (AstraZeneca/MedImmune), a human immunoglobulin (Ig) Glk monoclonal antibody that blocks PD-L1 binding to its receptors.

Agents that bind to the DNA or mRNA encoding PD-L1 also can act as PD-L inhibitors, e.g., small inhibitory anti-PD-L1 RNAi, small inhibitory anti-PD-L1 RNA, anti-PD-L1 anti-sense RNA, or dominant negative PD-L1 protein. Antagonists of or agents that antagonize PD-L1, e.g., anti-PD-L1 antibodies and PD-L1 antagonists, may include, but are not limited to those previously mentioned and any of those that are disclosed in Stewart et al., 2015, 3 (9): 1052-62; Herbst et al., 2014, Nature Volume: 515: Pages: 563-567; Brahmer et al., N Engl J Med 2012; 366:2455-2465; U.S. Pat. No. 8,168,179; US20150320859; and/or US20130309250 all incorporated herein by reference. In clinical trials, treatment with anti-PD-L1 antibodies resulted in less adverse events than did treatment with anti-PD-1 antibodies (Shih et al., 2014). In some embodiments, anti-PD-L1 inhibitors be used for treatment in combination with additional immune checkpoint blockade, e.g., with anti-PD-1, anti-CTLA-4, and/or anti-LAG-3 inhibitors.

b. Programmed Cell Death Protein 1 (PD-1)

In humans, programmed cell death protein 1 (PD-1) is encoded by the PDCD1 gene. PDCD1 has also been designated as CD279 (cluster of differentiation 279). This gene encodes a cell surface membrane protein of the immunoglobulin superfamily. PD-1 is a 288 amino acid cell surface protein molecule. PD-1 is expressed on the surface of activated T cells, B cells, and macrophages. PD-1 is expressed in pro-B cells and is thought to play a role in their differentiation. See T. Shino hara et al., Genomics 23 (3): 704-6 (1995). PD-1 is a member of the extended CD28CTLA-4 family of T cell regulators. (Y. Ishida et al., “EMBO J. 11 (11): 3887-95, (1992)). PD-1 may negatively regulate immune responses. PD-1 limits autoimmunity and the activity of T cells in peripheral tissues at the time of an inflammatory response to infection.

PD-1 has two ligands, PD-L1 and PD-L2, which are members of the B7 family. PD-L1 protein is upregulated on macrophages and dendritic cells (DC) in response to lipopolysaccharide (LPS) and granulocyte-macrophage colony-stimulating factor (GM-CSF) treatment, and on T cells and B cells upon T cell receptor (TCR) and B cell receptor signaling, whereas in resting mice, PD-L1 mRNA can be detected in the heart, lung, thymus, spleen, and kidney. PD-L1 is expressed on almost all murine tumor cell lines, including PA myeloma, PH815 mastocytoma, and B16 melanoma upon treatment with IFN-y. PD-L1 has been found to be highly expressed by several cancers and several PD-1 antagonists are being developed or are approved for treatment of cancer. PD-L2 expression is more restricted and is expressed mainly by DCs and a few tumor lines.

Programmed Death 1 (PD-1) antagonist. A PD-1 antagonist is a molecule that binds to PD-1 protein or to a gene or nucleic acid encoding PD-1 protein and inhibits or prevents PD-1 activation. Without wishing to be bound by theory, it is believed that such molecules reduce or block the interaction of PD-1 with its ligand(s) PD-L1 and/or PD-L2. PD-1 activity may be interfered with by antibodies that bind selectively to and block the activity of PD-1. The activity of PD-1 can also be inhibited or blocked by molecules other than antibodies that bind PD-1. Such molecules can be small molecules or can be peptide mimetics of PD-L1 and PD-L2 that bind PD-1 but do not activate PD-1. Molecules that antagonize PD-1 activity include those described in U.S. Publications 20130280265, 20130237580, 20130230514, 20130109843, 20130108651, 20130017199, 20210008200 and 20120251537, 20110271358, EP 2170959B1, the entire disclosures of which are incorporated herein by reference. See also M. A. Curran, et al., Proc. Natl. Acad. Sci. USA 107, 4275 (2010); S. L. Topalian, et al., New Engl. J. Med. 366, 2443 (2012); J. R. Brahmer, et al., New Engl. J. Med. 366, 2455 (2012); and D. E. Dolan et al., Cancer Control 21, 3 (2014), all incorporated by reference herein, in their entireties.

Herein, exemplary PD-1 antagonists include: nivolumab, also known as BMS-936558, OPDIVO® (Bristol-Meyers Squibb, and also known as MDX-1106 or ONO 4538), a fully human IgG4 monoclonal antibody against PD-1; pidilizumab, also known as CT-011 (CureTech), a humanized IgG1 monoclonal antibody that binds PD-1; MK-3475 (Merck, and also known as SCH 900475), an IgG4 antibody that binds PD-1; and pembrolizumab (Merck, also known MK-3475, lambrolizumab, KEYTRUDA®), a humanized IgG4-kappa monoclonal antibody that binds PD-1; MEDI-0680 (AstraZeneca/MedImmune), a monoclonal antibody that binds PD-1; and REGN2810 (Regeneron/Sanofi), a monoclonal antibody that binds PD-1. Another exemplary PD-1 antagonist is AMP 224 (Glaxo Smith Kline and Amplimmune), a recombinant fusion protein composed of the extracellular domain of the PD-1 ligand programmed cell death ligand 2 (PD-L2) and the Fc region of human IgG1, that binds to PD-1. Agents that interfere and bind to the DNA or mRNA encoding PD-1 also can act as PD-1 inhibitors. Examples include a small inhibitory anti-PD-1 RNAi, an anti-PD-1 antisense RNA, or a dominant negative protein. PDL-2 fusion protein AMP-224 (codeveloped by Glaxo Smith Kline and Amplimmune) is believed to bind to and block PD-1. In some embodiments, anti-PD-1 inhibitors may be used for treatment in combination with additional immune checkpoint blockade, e.g., with anti-PD-L1, anti CTLA-4, and/or anti-LAG-3 inhibitors.

c. Cytotoxic T-Lymphocyte-Associated Protein 4 (CTLA-4)

CTLA-4 (also known as CTLA-4 or cluster of differentiation 152 (CD152)), is a transmembrane glycoprotein that, in humans, is encoded by the CTLA-4 gene. CTLA-4 is a member of the immunoglobulin superfamily, which is expressed on the surface of helper T cells and is present in regulatory T cells, where it may be important for immune function. CTLA-4, like the homologous CD28, binds to B7 molecules, particularly CD80/B7-1 and CD86/B7-2 on antigen-presenting cells (APCs), thereby sending an inhibitory signal to T cells. CTLA-4 functions as an immune checkpoint that inhibits the immune system and is important for maintenance of immune tolerance.

CTLA-4 antagonist. A CTLA-4 antagonist, as used herein, is a molecule that binds to CTLA-4 protein or to a gene or nucleic acid encoding CTLA-4 protein and inhibits or prevents CTLA-4 activation. Without wishing to be bound by theory, it is believed that such molecules reduce or block the interaction of CTLA-4 with its ligands, e.g., B7 molecules CD80/B7-1 and CD86/B7-2. CTLA-4 activity may be blocked by molecules that bind selectively to and block the activity of CTLA-4 or that bind selectively to its counter-receptors, e.g., CD80, CD86, etc. and block activity of CTLA-4. Blocking means inhibit or prevent the transmission of an inhibitory signal via CTLA-4. In some embodiments, anti-CTLA-4 antibodies may be used for treatment in combination with additional immune checkpoint blockade, e.g., with anti-PD-1, anti-PD-L1, and/or anti-LAG-3 treatment. CTLA-4 antagonists include, for example, inhibitory antibodies directed to CD80, CD86, and/or CTLA-4; small molecule inhibitors of CD80, CD86, and CTLA-4; antisense molecules directed against CD80, CD86, and/or CTLA-4; adnectins directed against CD80, CD86, and/or CTLA-4; and RNAi inhibitors (both single and double stranded) of CD80, CD86, and/or CTLA-4. Suitable CTLA-4 antagonists and/or anti-CTLA-4 antibodies include humanized anti-CTLA-4 antibodies, such as MDX-010/ipilimumab (Bristol-Meyers Squibb), tremelimumab/CP-675,206 (Pfizer, AstraZeneca), and antibodies that are disclosed in PCT Publication No. WO 2001/014424, PCT Publication No. WO 2004/035607, U.S. Publication No. 2005/0201994, European Patent No. EP 1212422 B1, U.S. Pat. Nos. 5,811,097, 5,855,887, 6,051,227, 6,984,720, 7,034,121, 8,475,790, U.S. Publication Nos. 2002/0039581 and/or 2002/086014, the entire disclosures of which are incorporated herein by reference.

d. Lymphocyte-Activation Gene 3 (LAG-3)

The term “LAG-3” or “LAG3” refers to Lymphocyte Activation Gene-3. The LAG-3 protein, which belongs to immunoglobulin (Ig) superfamily, comprises a 503-amino acid type I transmembrane protein with four extracellular Ig-like domains, designated DI to D4. LAG-3 is an immune checkpoint protein that plays a role in regulating the immune system. As described herein, the term LAG-3 includes variants, isoforms, homologs, orthologs, and paralogs. For example, antibodies specific for a human LAG-3 protein may, in certain cases, cross-react with a LAG-3 protein from a species other than human. In other embodiments, the antibodies specific for a human LAG-3 protein may be completely specific for the human LAG-3 protein and may not exhibit species or other types of cross-reactivity or may cross-react with LAG-3 from certain other species but not all other species (e.g., cross-react with monkey LAG-3, but not mouse LAG-3). The term “human LAG-3” refers to human sequence LAG-3, such as the complete amino acid sequence of human LAG-3 having GenBank Accession No. NP 002277. LAG-3 is also known in the art as, for example, CD223. The human LAG-3 sequence may differ from human LAG-3 of GenBank Accession No. NP 002277 by having, e.g., conserved mutations or mutations in non-conserved regions and the LAG-3 has substantially the same biological function as the human LAG-3 of GenBank Accession No. NP 002277. For example, a biological function of human LAG-3 is having an epitope in the extracellular domain of LAG-3 that is specifically bound by an antibody of the instant disclosure or a biological function of human LAG-3 is binding to MHC Class II molecules.

LAG-3 Antagonists. Inhibitors of LAG-3 are immunotherapy treatments for various cancers and autoimmune diseases. One of the notable LAG-3 inhibitors that has been developed is Relatlimab. Relatlimab is a monoclonal antibody that targets LAG-3 and is designed to block its interaction with its ligand, MHC class II molecules, which can suppress immune responses. By inhibiting LAG-3, Relatlimab aims to enhance the activation and function of T cells, the immune cells responsible for recognizing and attacking cancer cells. Agents that interfere and bind to the DNA or mRNA encoding LAG-3 proteins also can act as LAG-3 inhibitors. Examples include a small inhibitory anti-LAG-3 RNAi, an anti-LAG-3 antisense RNA. In some embodiments, anti-LAG3 inhibitors may be used for treatment in combination with additional immune checkpoint blockade, e.g., with anti-PD-1, anti-PD-L1, and/or anti-CTLA-4 inhibitors.

### C. Treatment Following HCC Conversion

The disclosed triple therapy can be followed by a further treatment or treatments to ameliorate, put into remission, or cure the subject's HCC. For example, the disclosed triple therapy can be followed by resecting tumor tissue in the subject and/or by treating the subject with radiofrequency ablation.

i. Resection

Tumor resection, also known as “resection,” “tumor removal” is a surgical procedure in which a tumor or abnormal growth is removed or excised or resected from the affected region of a body. The tumor along with some healthy tissues nearby (also called margin) are also removed during the surgical procedure. This procedure is commonly used in the treatment of various types of cancer and non-cancerous tumors. The primary goal of tumor resection is to eliminate the tumor while minimizing damage to surrounding healthy tissues and organs. An organ or tissue may need to be removed for various reasons, including disease, damage, or trauma.

Tumor removal generally requires a larger incision, or cut, than a biopsy. There may be less invasive surgical options for tumor removal, like a laparoscopic surgery or robotic surgery. These use small instruments and small incisions. With a less invasive surgery, there is less pain and faster recovery.

Sometimes, resection is the only cancer treatment needed. Sometimes, additional treatments before or after resection is needed. Various treatments can be given before resection to help shrink tumor size. These treatments can combine chemotherapy, embolization, chemoembolization (such as TACE), radiation therapy (including SBRT), immunotherapy (such as immune checkpoint inhibitors), or any combination of these in any sequence or manner.

ii. Radiofrequency Ablation

Radiofrequency ablation, or RFA, is a minimally invasive technique that uses heat to kill cancer cells or shrink the size of tumors, nodules, or other growths in the body. RFA is used to treat a range of conditions, including benign and malignant tumors, chronic venous insufficiency in the legs, as well as chronic back and neck pain. The procedure is similar to a needle biopsy and involves inserting a needle-like probe into the body. Radiofrequency waves are sent out from the probe into the surrounding tissue, which causes the nearby cells to die. As these cells die, the immune system removes them, which causes an internal reaction and generally results in the shrinkage of the nodule. To place the tip of the probe in the precise location of tumor or abnormal tissue, the health care provider uses ultrasound or other imaging technique such as CT (computed tomography) or MRI (magnetic resonance imaging).

Radiofrequency ablation can take place in an office or outpatient setting and requires no general anesthesia. Subjects can obtain medicine to help relax for the procedure as well as a numbing agent for the area of the skin where the probe is inserted. Most subjects undergoing radiofrequency ablation can go home the same day as their treatment and can return to their normal activities within 24 hours.

RFA is not usually the main treatment for cancer. Various treatments for cancer, including HCC, can be administered before administering RFA to help shrink the tumor or kill cancerous cells. These treatments can combine chemotherapy, embolization, chemoembolization (such as TACE), radiation therapy (including SBRT), immunotherapy (such as immune checkpoint inhibitors), or any combination of these in any sequence or manner.

iii. Liver Transplant

“Liver transplant”, “liver transplantation”, or “hepatic transplantation” is a surgical procedure in which a diseased or damaged liver is replaced with a healthy liver from a deceased or living donor. It is a lifesaving treatment for individuals with end-stage liver disease, acute liver failure, or certain types of liver cancer that cannot be effectively treated by other means.

It is recommended to have a liver transplant for subjects with early liver cancer, especially those who also have severe cirrhosis, or scarring of the organ. A transplant is usually reserved for subjects with liver cancer who have 1 tumor that is up to 5 centimeters in diameter, or 2 or 3 tumors that are each less than 3 centimeters in diameter. A transplant is usually not an option when the cancer has metastasized or spread to other parts of the body. Subjects having cancer, including those with locally advanced HCC, can also be eligible for liver transplants after conversion therapies that reduce tumor size and number of tumors. This conversion therapy can combine chemotherapy, embolization, chemoembolization (such as TACE), radiation therapy (including SBRT), immunotherapy (such as immune checkpoint inhibitors), or any combination of these in any sequence or manner.

### 1. Sequence of Triple Treatment

Sequence of triple treatment as described herein refers to a method of treating subjects with locally advanced unresectable hepatocellular carcinoma (HCC) that comprises treating the subject with (a) Transarterial Chemoembolization (TACE), (b) Stereotactic body radiotherapy (SBRT), and (c) Immune checkpoint inhibitors sequentially.

The terms “high,” “higher,” “increases,” “elevates,” or “elevation” refer to increases above basal levels, e.g., as compared to a control. The terms “low,” “lower,” “reduces,” or “reduction” refer to decreases below basal levels, e.g., as compared to a control.

The term “modulate” as used herein refers to the ability of a compound to change an activity in some measurable way as compared to an appropriate control. As a result of the presence of compounds in the assays, activities can increase or decrease as compared to controls in the absence of these compounds. Preferably, an increase in activity is at least 25%, more preferably at least 50%, most preferably at least 100% compared to the level of activity in the absence of the compound. Similarly, a decrease in activity is preferably at least 25%, more preferably at least 50%, most preferably at least 100% compared to the level of activity in the absence of the compound. A compound that increases a known activity is an “agonist.” One that decreases, or prevents, a known activity is an “antagonist.”

The term “inhibit” means to reduce or decrease in activity or expression. This can be a complete inhibition of activity or expression, or a partial inhibition. Inhibition can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64,65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

The term “monitoring” as used herein refers to any method in the art by which an activity can be measured.

The term “providing” as used herein refers to any means of adding a compound or molecule to something known in the art. Examples of providing can include the use of pipettes, pipettemen, syringes, needles, tubing, guns, etc. This can be manual or automated. It can include transfection by any mean or any other means of providing nucleic acids to dishes, cells, tissue, cell-free systems and can be in vitro or in vivo.

The term “preventing” as used herein refers to administering a compound prior to the onset of clinical symptoms of a disease or conditions so as to prevent a physical manifestation of aberrations associated with the disease or condition.

The term “in need of treatment” as used herein refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that include the knowledge that the subject is ill, or will be ill, as the result of a condition that is treatable by the disclosed compounds.

As used herein, “subject” includes, but is not limited to, animals, plants, bacteria, viruses, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects. For the disclosed methods, preferred subjects are mammals, more preferred are humans and domesticated animals, especially preferred are humans.

By “treatment” and “treating” is meant the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, amelioration, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount. For the disclosed methods, preferred courses of treatment (e.g., the disclosed triple therapy) include applying/administering the treatments to a subject with the intent to cure, convert, ameliorate, or stabilize, HCC, more preferably with the intent to convert the HCC.

By the term “effective amount” of a compound as provided herein is meant a nontoxic but sufficient amount of the compound to provide the desired result. As will be pointed out below, the exact amount required will vary from subject to subject, depending on the species, age, and general condition of the subject, the severity of the disease that is being treated, the particular compound used, its mode of administration, and the like. Thus, it is not possible to specify an exact “effective amount.” However, an appropriate effective amount can be determined by one of ordinary skill in the art using only routine experimentation.

The dosages or amounts of the compounds should be large enough to produce the desired effect in the method by which delivery occurs. The dosage should not be so large as to cause adverse side effects, such as unwanted cross-reactions, anaphylactic reactions, and the like. Generally, the dosage will vary with the age, condition, sex and extent of the disease in the subject and can be determined by one of skill in the art. The dosage can be adjusted by the individual physician based on the clinical condition of the subject involved. The dose, schedule of doses and route of administration can be varied.

The efficacy of administration of a particular dose of the compounds or compositions according to the methods described herein can be determined by evaluating the particular aspects of the medical history, signs, symptoms, and objective laboratory tests that are known to be useful in evaluating the status of a subject having HCC. These signs, symptoms, and objective laboratory tests will generally be known to any clinician who treats such patients.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

It is to be understood that the disclosed compounds, compositions, and methods are not limited to specific synthetic methods, specific analytical techniques, or to particular reagents unless otherwise specified, and, as such, may vary. It is also to be understood that the terminology used herein is for the purpose of describing particular forms and embodiments only and is not intended to be limiting.

The disclosed compositions and methods can be further understood through the following numbered paragraphs.

1. A method for treating a subject having hepatocellular carcinoma (HCC), the method comprising:
(a) treating the subject with transarterial chemoembolization (TACE);
(b) treating the subject with stereotactic body radiotherapy (SBRT); and
(c) treating the subject with an immune checkpoint inhibitor, wherein the HCC comprises one or more tumors.

2. The method of paragraph 1, wherein the subject's HCC is locally advanced.

3. The method of paragraph 1 or 2, wherein the subject's HCC is unresectable.

4. The method of any one of paragraphs 1-3, wherein steps (a), (b), and (c) are performed sequentially in that order.

5. The method of any one of paragraphs 1-4, wherein the immune checkpoint inhibitor targets programmed cell death ligand 1 (PD-L1).

6. The method of any one of paragraphs 1-4, wherein the immune checkpoint inhibitor targets programmed cell death protein 1 (PD-1).

7. The method of any one of paragraphs 1-4, wherein the immune checkpoint inhibitor targets cytotoxic T-lymphocyte-associated protein 4 (CTLA-4).

8. The method of any one of paragraphs 1-4, wherein the immune checkpoint inhibitor targets lymphocyte-activation gene 3 (LAG-3).

9. The method of any one of paragraphs 1-8, wherein the immune checkpoint inhibitor comprises a therapeutic antibody.

10. The method of paragraph 9, wherein the therapeutic antibody is avelumab, atezolizumab, durvalumab, envafolimab, cosibelimab, or AUNP-12.

11. The method of paragraph 9, wherein the therapeutic antibody is pembrolizumab, nivolumab, cemiplimab, spartalizumab, camrelizumab, sintilimab, tislelizumab, toripalimab, dostarlimab, retifanlimab, AMP-224, or MEDI0680.

12. The method of paragraph 9, wherein the therapeutic antibody is ipilimumab or tremelimumab.

13. The method of paragraph 9, wherein the therapeutic antibody is relatlimab.

14. The method of any one of paragraphs 1-14, wherein TACE comprises infusing a chemotherapeutic agent and an embolic particle into an artery supplying blood to one or more tumors of the HCC.

15. The method of paragraph 14, wherein the chemotherapeutic agent and the embolic particle are infused together or separately.

16. The method of paragraph 14 or 15, wherein the chemotherapeutic agent and the embolic particle are infused sequentially.

17. The method of any one of paragraphs 14-16, wherein the embolic particle comprises a polyvinyl alcohol microsphere, superabsorbent microsphere, gelatin microsphere, or degradable starch microsphere.

18. The method of any one of paragraphs 1-17 further comprising:
resecting tumor tissue in the subject.

19. The method of any one of paragraphs 1-18 further comprising:
treating the subject with radiofrequency ablation.

### EXAMPLES

### Example 1: Development and Demonstration of START-FIT Triple Therapy

### Methods

### Study Design and Participants

START-FIT was a single-arm, multicentre, investigator-initiated phase 2 trial that investigated the activity and safety of sequential TACE, stereotactic body radiotherapy, and avelumab (an anti-PD-L1 drug) in patients with locally advanced hepatocellular carcinoma, and was conducted at Queen Mary Hospital (Pokfulam, Hong Kong Special Administrative Region, China), Tuen Mun Hospital (Tuen Mun, Hong Kong Special Administrative Region, China), and University of Hong Kong-Shenzhen Hospital (Shenzhen, China). Patients aged 18 years or older with unresectable hepatocellular carcinoma without lymph node or extrahepatic metastases were eligible. Tumors were classified as unresectable after a multidisciplinary team review because either: (1) RO resection was not feasible; (2) remnant liver volume was less than 30% in patients who did not have cirrhosis or 40% in patients with cirrhosis, or the results of an indocyanine green test were higher than 15%; (3) patients had Barcelona Clinic Liver Cancer (BCLC) stage B and beyond Up-to-7 criteria; or (4) patients had BCLC stage C. Other major inclusion criteria included Eastern Cooperative Oncology Group performance status 0-1; Child-Pugh liver function score A5 to B7; tumor size of at least 5 cm; a maximum of three tumor lesions; and adequate hepatic, renal, and bone marrow functions. Previous curative treatment (e.g., resection, radiofrequency ablation, percutaneous ethanol injection) was allowed. Tumors with branched portal vein (VP1 to VP3) or hepatic vein invasion (VV1 to VV2) were allowed. Patients with distant metastasis or tumors with main portal vein invasion (VP4) or inferior vena cava (VV3) were excluded. Patients with severe comorbidities (eg, symptomatic congestive heart failure, unstable angina, and uncontrolled hypertension) or an estimated life expectancy of less than 3 months were excluded. Patients who received any previous systemic therapy, TACE, radiotherapy to the liver, selective internal radiation, or those with liver volume minus the gross tumor volume of 700 mL or less were also excluded. A comprehensive list of eligibility criteria can be found in the Tables 2A and 2B. All patients provided written informed consent, and the institutional review board committee approved the protocol (institutional review board number: UW 18-541). The study was conducted in accordance with the Declaration of Helsinki and the Good Clinical Practice standards.

### Table 2A: Inclusion Criteria

1. Patients with hepatocellular carcinoma (HCC) that is not suitable for resection, liver transplant, or ablation treatment who are planned for TACE as per institutional practice
2. Diagnosis of HCC confirmed by histology/cytology or clinically by AASLD criteria in cirrhotic subjects. Patients without cirrhosis require histological confirmation of diagnosis.
3. Patients must have at least one measurable lesion according to RECIST v1.1.
4. Male or female subjects aged ≥18 years old
5. Eastern Cooperative Oncology Group (ECOG) performance status score of 0 or 1
6. At least one liver nodule with size ≥5 cm and number of lesions ≤3
7. Intrahepatic vascular invasion includes intrahepatic portal vein branch invasion, left or right portal vein invasion, and main hepatic vein invasion.
8. Child-Pugh liver function class A-B7
9. Liver volume minus intrahepatic GTV>700 cc
10. No prior radiotherapy or radioembolization to the liver or upper abdomen was allowed. Prior local therapies, such as surgery, radiofrequency ablation, percutaneous ethanol injection or cryoablation, are allowed if the index lesion(s) remain outside of the treatment field or have progressed since prior treatment. Local therapy must have been completed at least 4 weeks prior to the baseline scan.
11. Adequate organ and marrow function, as defined below:
   a) Hemoglobin ≥9 g/dL
   b) Absolute neutrophil count ≥1,500/μL
   c) Platelet count ≥100,000/μL
   d) Total bilirubin ≤2.0×ULN
   e) ALT≤3×ULN
   f) Albumin ≥2.8 g/dL
   g) INR ≤1.6
   h) Calculated creatinine clearance ≥45 mL/minute as determined by Cockcroft-Gault (using actual body weight) or 24-hour urine creatinine clearance

### Table 2B: Exclusion Criteria

1. Patients were excluded if they were receiving any other investigational agents or using an investigational device within 4 weeks of the first dose of treatment. Patients are excluded if they are receiving other systemic therapy within 2 weeks of the first dose of treatment.
2. Prior invasive malignancy within 2 years except for noninvasive malignancies such as cervical carcinoma in situ, in situ prostate cancer, non melanomatous carcinoma of the skin, lobular or ductal carcinoma in situ of the breast that has been surgically cured
3. Presence of extrahepatic metastases (M1)
4. Extrahepatic vascular involvement or thrombosis: main portal vein (Vp4) or inferior vena cava (IVC) (VV3)
5. Prior radiotherapy to the liver or upper abdomen
6. Hepatic encephalopathy, active or untreated gastrointestinal varices
7. Untreated central nervous system (CNS) metastatic disease, leptomeningeal disease, or cord compression
8. Uncontrolled intercurrent illness including but not limited to, ongoing or active infection, symptomatic congestive heart failure, unstable angina pectoris, cardiac arrhythmia, uncontrolled hypertension, or psychiatric illness/social situations that would limit compliance with study requirements.
9. Prior treatment with any other anti-programmed cell death protein 1 (anti-PD-1), PD ligand-1 (PD-L1) or PD ligand-2 (PD-L2) agent or an antibody targeting other immunoregulatory receptors or mechanisms
10. Irritable bowel syndrome or other serious gastrointestinal chronic conditions associated with diarrhea within the past 3 years prior to the start of treatment
11. Known history of testing positive for HIV or known acquired immunodeficiency syndrome.
12. On chronic systemic steroid or any other forms of immunosuppressive medication within 14 days prior to the treatment. Except:
   (a) Intranasal, inhaled, topical steroids, or local steroid injection (e.g. intra articular injection).
   (b) Systemic corticosteroids at physiologic doses ≤10 mg/day of prednisone or equivalent.
   (c) Steroids as premedication for hypersensitivity reactions (e.g. CT scan premedication).
13. Active or prior documented autoimmune or inflammatory disorders in the past 2 years, except diabetes type I, vitiligo, psoriasis, or hypo or hyperthyroid diseases not requiring immunosuppressive treatment
14. History of primary immunodeficiency or solid organ transplantation
15. Receipt of live, attenuated vaccine within 28 days prior to the study treatment
16. Severe hypersensitivity reaction to treatment with another monoclonal antibody (mAb)
17. Females who are pregnant, lactating, or intend to become pregnant during their participation in the study

### Procedures

All patients underwent a single treatment of conventional TACE within 28 days of study enrolment. Conventional TACE was performed by supra-selective cannulation of all the branches supplying the tumor. The emulsion was prepared by mixing iodised oil with cisplatin (1 mg/mL) in a 1:1 ratio. A maximum of 60 mL of emulsion was injected. Preparation of the emulsion was followed by embolisation with gelfoam (gelatin granules) pellets of 1 mm diameter mixed with 40 mg of gentamicin. At 28 days (plus or minus 3 days) after the completion of TACE, stereotactic body radiotherapy was delivered to all lesions. Patients were immobilised via a vacuum foam bag (Vac-Lok™; MEDTEC, IA, USA) and active breathing control to reduce liver motion. Imaging was performed on the inhale breath-hold contrast CT. Gross tumor volume was defined as a tumor focus that was visualised by contrast imaging. The clinical target volume was defined as gross tumor volume with expansion to include the area stained with iodised oil. The individualised planning target volume margins were formulated to compensate for respiratory motion and setup errors. Cone beam CT was acquired on board before each treatment. A radiation dose of 27.5-40.0 Gy in five fractions delivered daily was allowed per protocol. The prescription isodose encompassed 95% of the planning target volume. The final dose was determined such that a maximum tumoricidal dose could be delivered to tumors while respecting the tolerance dose of the organ at risk (Table 3). The radiotherapy toxicity was assessed by CTCAE; version 4.01.

TABLE 3
Dose constraints of major organ-at-risk (OARs)

| | |
|---|---|
| Ideal | Acceptable |

Mandatory dose constraints

| | | |
|---|---|---|
| Liver (Liver-GTV) mean dose | | |
| 40 Gy* | ≤15 Gy | 15-15.2 Gy |
| 35 Gy* | ≤15.5 Gy | 15.5-15.7 Gy |
| 30 Gy* | ≤16 Gy | 16-16.2 Gy |
| 27.5 Gy* | ≤17 Gy | 17-17.2 Gy |
| Liver-GTV: V10 | <70% | |
| Liver-GTV | >700 cc | |
| Stomach, small intestine, duodenum: | 30 Gy | >30 Gy but ≤32 Gy |
| Max (to 0.5 cc) | | |
| Large bowel, esophagus: | 32 Gy | >32 Gy but ≤34 Gy |
| Max (to 0.5 cc) | | |
| Spinal cord + 5mm: | 25 Gy | >25 Gy but ≤28 Gy |
| Max (to 0.5 cc) | | |
| Kidney: Bilateral mean dose | ≤10 Gy | >10 Gy but ≤12 Gy |

Non-mandatory dose constraints

| | |
|---|---|
| Heart: Max (to 30 cc) | <30 Gy |
| Gall bladder: Max (to 0.5 cc) | <55 Gy |
| Common bile duct: Max (to 0.5 cc) | <50 Gy |
| Central biliary structure | V40 <21 cc |
| Chest wall | V30 <30 cc |
| Great vessel: Max (to 0.5 cc) | <60 Gy |

*Prescription dose to planning target volume (PTV)
Abbreviations:
GTV: gross tumor volume

Patients received the first dose of intravenous avelumab (10 mg/kg) 14 days (plus or minus 3 days) after the completion of stereotactic body radiotherapy. Avelumab was then given every 2 weeks until the development of grade 3 or worse immune-related adverse events, disease progression, or withdrawal of consent. If the tumor was deemed amenable to surgical intervention, avelumab was also stopped. Dose reduction was not allowed. Dose interruption was permitted up to a maximum of 12 weeks. There was no requirement for the minimum treatment duration of avelumab.

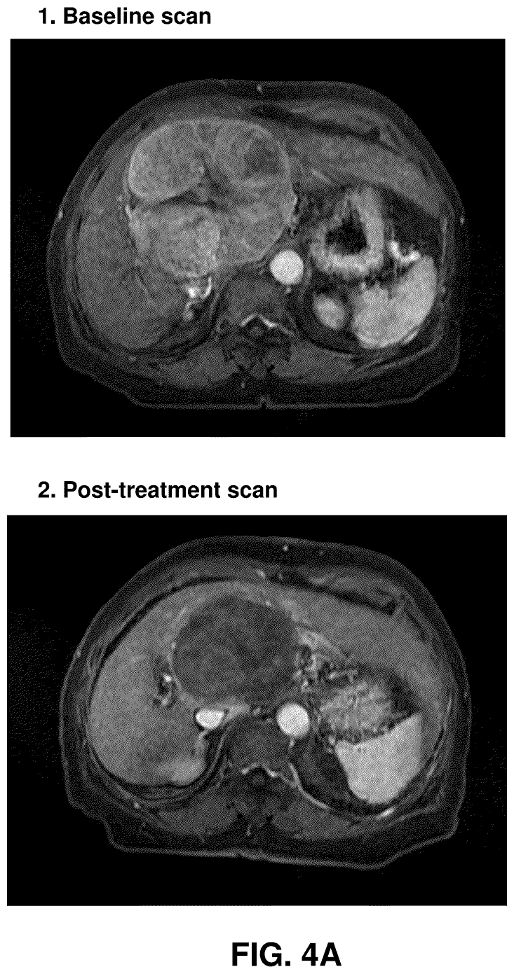

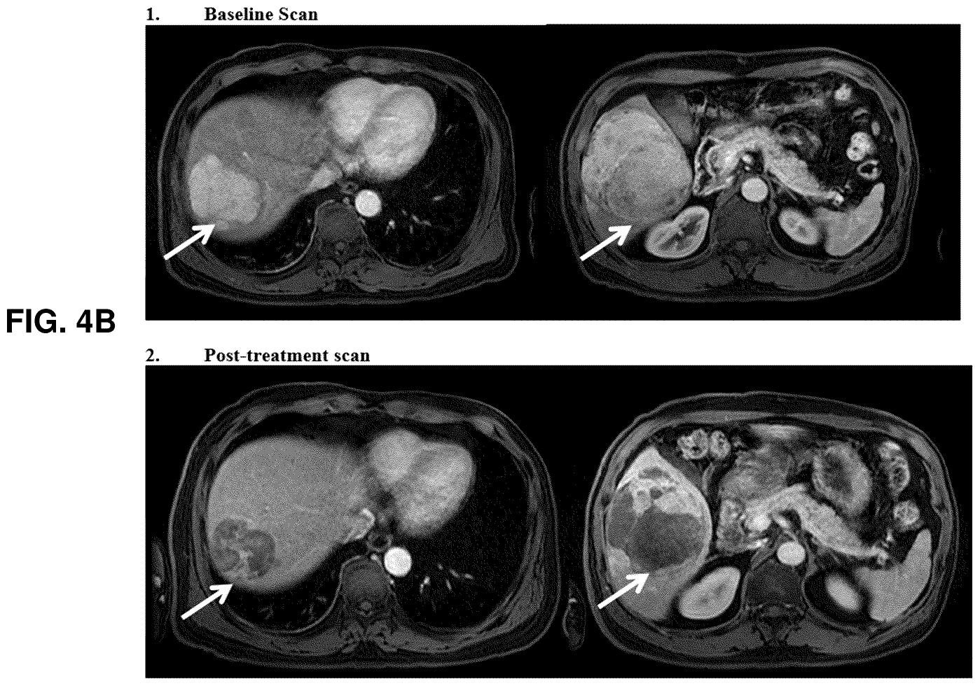

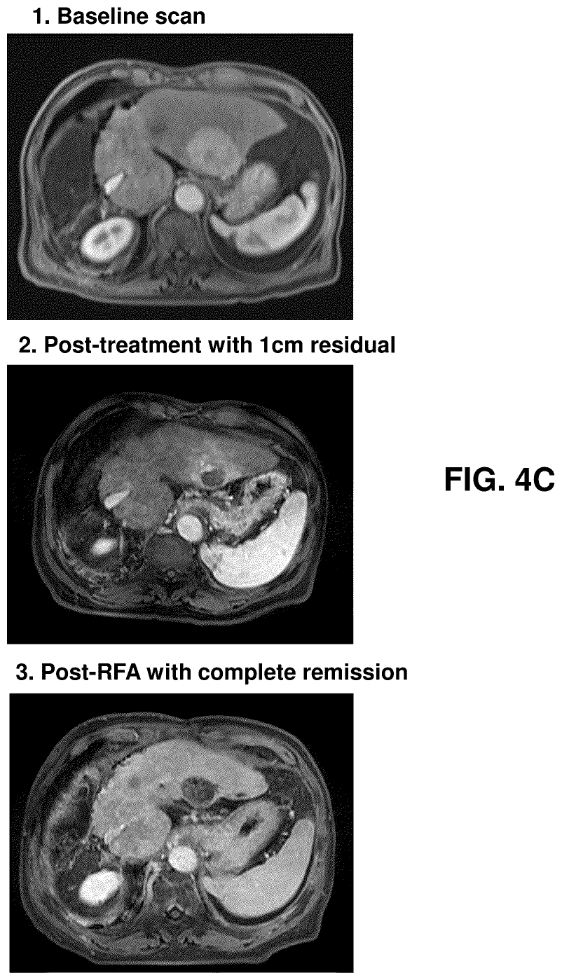

All patients underwent baseline contrast-enhanced MRI of the liver and CT of the thorax. Treatment response evaluation was assessed with MRI after cycle 4, cycle 8, and cycle 12 of avelumab and every 12 weeks thereafter (plus or minus 7 days). Radiological imaging was reported using the modified Response Evaluation Criteria in Solid Tumors (mRECIST). A CT of the thorax was done every 26 weeks or at the time of radiological or clinical evidence of disease progression according to mRECIST criteria. All patients were discussed in a multidisciplinary team board meeting of surgeons specializing in liver transplantation and liver surgery, clinical oncologists, interventional radiologists, and diagnostic radiologists. This meeting was held once every 2 weeks to decide resectability and treatment failure. All patients underwent complete tumor staging to exclude distant metastasis before surgery. A board-certified radiologist conducted an independent blinded review in addition to the investigator review. Safety assessments were documented throughout the treatment period. Adverse events were graded according to the US National Cancer Institute Common Terminology Criteria for Adverse Events (CTCAE; version 4.01). The frequency, duration, and severity of adverse events were recorded. An example of the resultant MRIs are shown in FIG. 4.

For biomarker assessment, AFP concentration and albumin-bilirubin score were measured at baseline, before stereotactic body radiotherapy, before avelumab initiation, every 2 weeks during the first 6 months of treatment with avelumab, and every 4 weeks thereafter. Exosomal PD-L1 concentration was measured at baseline, before stereotactic body radiotherapy, before avelumab initiation, and every 2 weeks in the first 3 months of treatment with avelumab.

Blood samples (total 20 mL) were collected in EDTA Vacutainer tubes (BD Vacutainer blood collection tubes) at each time point and processed within 4 hours of collection. Cell free plasma will be first prepared from a 10 ml blood sample and then centrifuged at 16,500×g for 45 min (5418R) to pellet large membrane vesicles. The supernatant will be passed through 0.22 μM filters. Extracellular vesicles will then be purified from the supernatants using the exosome isolation kit (ExoQuick® ULTRA EV Isolation Kit for Serum and Plasma, cat #EQULTRA 20A 1). The purified extracellular vesicles will be incubated with 10 μl CD63 coated magnetic beads (Miltenyi Biotech, Cat #130 110 918 or Invitrogen, Cat #10606D) in 100 μl peripheral blood samples with 0.1% bovine serum albumin (overnight at 4 C with mixing. Exo some coated beads will then be washed and incubated with fluorophore labeled PD L1 antibodies, followed by analysis on an LSR II flow cytometer (BD).

To test the binding of exosomal PD-L1 to PD-1, 100 μl of exosome samples of different concentrations will be captured onto PD-L1 antibody-coated 96-well ELISA plates by overnight incubation at 4° C. Then, 100 ul of 4 μg/ml biotin-labeled human PD-1 protein (BPS Bioscience, Cat #71109) will be added and incubated for 1 hour at room temperature. A total of 100 μl per well of horseradish peroxidase-conjugated streptavidin (BD) diluted in peripheral blood sample containing 0.1% BSA will then added and incubated for 1 hour at room temperature. Plates will be read at 450 nm with a BioTek plate reader and compared against the prespecified positive and negative controls.

### Outcomes

The primary endpoint was the percentage of patients deemed to be amenable to curative treatment after conversion therapy. Amenability for curative treatment was fulfilled when one of the following criteria was met: either a sustained complete response or sustained partial response achieved for at least 2 months and if curative treatment could be performed (based on investigator's review). Curative treatment included RO resection, if sufficient liver volume and function could be retained; radiofrequency ablation, which was reserved for patients with tumors downsized to less than 3 cm for whom resection was not feasible due to tumor location, or patients with tumors in a superficial location that could be safely dealt with by percutaneous ablation;¹⁶ or transplantation, which was limited to patients with tumors downstaged to be within the University of California San Francisco criteria for liver transplantation that were deemed unsuitable for resection or ablation, who were 70 years or younger with cirrhosis complicated by portal hypertension.¹⁷ The secondary endpoints were: objective response rate according to modified RECIST; number of patients who became amenable and received curative treatment or achieved radiological complete response; progression-free survival, defined as time from TACE to first documented disease progression according to modified RECIST or death from any cause; time to progression, defined as time from TACE to first documented disease progression according to modified RECIST; overall survival, defined as time from TACE to date of death from any cause; quality-of-life measurement using the FACT-Hep score and EORTC QLQ-C30, measured every 3 months in the first year; toxicity as measured by the CTCAE (version 4.01) and Child-Pugh liver function score progression of two or more points;¹⁸ pathological response, defined as the percentage of surface with non-viable cancer cells in relation to the total tumor area; disease control rate (DCR), expressed as the percentage of patients who had a complete response, partial response, or stable disease for at least 6 months; local control rate, defined as the percentage of lesions with absence of recurrence within the high-dose region (80% isodose volume); duration of response, defined as time from first documented evidence of complete response or partial response until the first documented disease progression or death from any cause; pattern of disease progression (in-field, out-field intrahepatic, new vascular invasion, or extrahepatic) per modified RECIST; and radiological response per RECIST (version 1.1). Post-hoc analyses included radiological response according to immune RECIST, correlation of clinical outcomes with BCLC stage, albumin-bilirubin score, radiological assessment criteria, biomarkers, and median time to treatment response.

### Statistical Analysis

The sample size in this study was based on the assumption that approximately 20% of patients would be amenable to surgery with the START-FIT regimen, and that 5% of patients would be amenable to surgery with TACE only. These figures were extrapolated from historical institutional data. A modified Simon two-stage optimal design was used in view of its ability to minimize the expected number of patients under the null hypothesis, (80% power; level of significance, p=0.05; historical response rate, 5%; target response rate for treatment efficacy, 20%; stage 1 sample size of ten patients; total sample size of 29 patients, with an additional four patients to allow for dropout or other reasons).¹⁹ In the first stage, ten patients were enrolled. If at least one patient could proceed to surgery, an additional 19 patients were recruited. If at least four of the total 29 patients could proceed to surgery, the treatment combination would be considered worthy of further investigation. Assuming a 10% loss to follow-up, a total of 33 patients would be recruited. All patients who received any study treatment were included in the intention-to-treat population and analysed for the primary and secondary outcomes, including the safety outcomes.

The percentage of patients deemed amenable to treatment with curative intent and the corresponding 95% CI were expressed as a binomial distribution. The objective response rate and corresponding 95% CI were estimated using the Clopper-Pearson method. The survival distributions were estimated using the Kaplan-Meier method for all the time-to-event endpoints and compared between subgroups (albumin-bilirubin and BCLC) by the log-rank test. The Brookmeyer and Crowley method estimated the median time to an event and corresponding 95% CI.²⁰ Comparison of exosomal PD-L1 concentrations among complete responders versus non-complete responders was conducted at baseline and 1 month after immunotherapy. For overall survival, data for patients who were not known to have died at the time of the analysis were censored at the last recorded date that the patient was known to be alive. For progression-free survival, data for patients who had not had a progression event or had not died at the time of the analysis were censored at the time of their last assessment (according to mRESIST) that could be evaluated.

All quality of life scores were calculated using the EORTC and FACT-Hep methods, and the mean quality-of-life with 95% CI at each timepoint was tabulated. Analyses of outcomes between subgroups of patients with specific baseline categorical and continuous variables were conducted using a χ² distribution or a Mann-Whitney U test when appropriate. Statistical significance was defined as a p value of less than 0.05, and all the performed tests were two-tailed. Data were analysed using R (version 3.25). There was no data monitoring committee. This study is registered with ClinicalTrials.gov (NCT03817736).

### Results

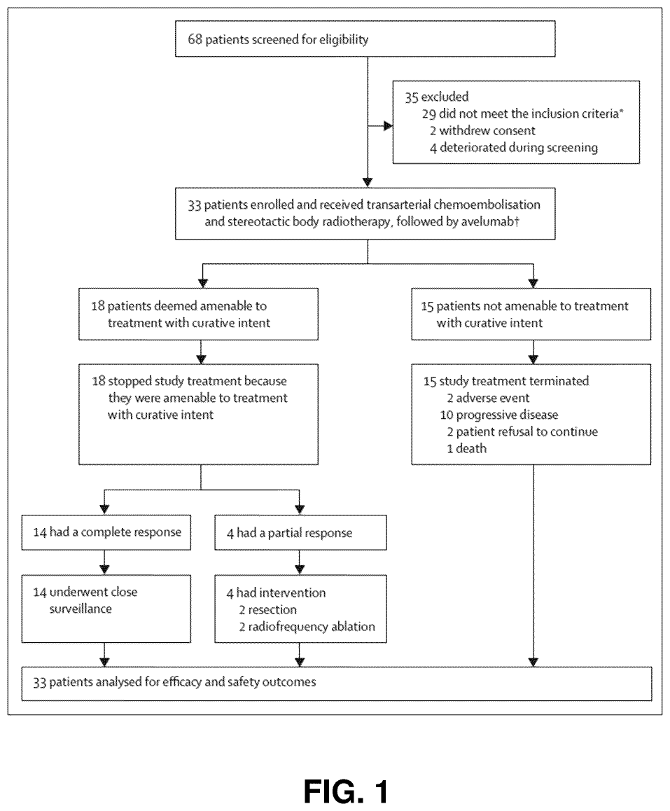

68 patients were screened for eligibility. 29 patients did not meet the eligibility criteria (FIG. 1). However, four patients had clinical deterioration before receiving the starting dose, and two patients withdrew consent (FIG. 1). Overall, 33 (49%) of 68 screened patients were deemed eligible and enrolled in the study. The baseline characteristics of the intention-to-treat population are detailed in Table 4. Most of the patients recruited were male and had Child-Pugh class A.²¹ (64%) patients had BCLC stage C disease with macrovascular invasion and eight had BCLC stage B disease and were beyond the up-to-7 criteria. The demographics and clinical characteristics of individual patients are presented in the Table 5 and Table 6.

TABLE 4
Baseline demographic and tumor characteristics
Patients (n = 33)

| | |
|---|---|
| Median age, years (range) | 68 (62-74.5) |

Sex

| | |
|---|---|
| Male | 32 (97%) |
| Female | 1 (3%) |

Eastern Cooperative Oncology Group performance status

| | |
|---|---|
| 0 | 26 (79%) |
| 1 | 7 (21%) |

Cause of liver cirrhosis

| | |
|---|---|
| Hepatitis B | 24 (73%) |
| Hepatitis C | 4 (12%) |
| Alcohol misuse | 2 (6%) |
| Cryptogenic | 3 (9%) |

Child-Pugh score

| | |
|---|---|
| A5 | 23 (70%) |
| A6 | 9 (27%) |
| B7 | 1 (3%) |

Albumin-bilirubin score

| | |
|---|---|
| Grade 1 | 21 (64%) |
| Grade 2 | 12 (36%) |

BCLC stage*

| | |
|---|---|
| A | 4 (12%) |
| B | 8 (24%) |
| C without extrahepatic spread | 21 (64%) |

Reasons for cancer being unresectable

| | |
|---|---|
| Inadequate liver remnant volume, | 4 (12%) |
| poor indocyanine green test result, | |
| or both | |
| BCLC stage B and beyond the | 8 (24%) |
| up-to-7 criteria† | |
| BCLC stage C without | 21 (64%) |
| extrahepatic spread | |

Tumour vascular invasion

| | |
|---|---|
| No | 12 (36%) |
| Yes | 21 (64%) |
| Branched portal vein invasion | 3 (9%) |
| Hepatic vein invasion | 13 (39%) |
| Both portal vein and hepatic vein | 5 (15%) |
| invasion | |

Number of lesions

| | |
|---|---|
| One | 17 (52%) |
| Two | 12 (36%) |
| Three | 4 (12%) |
| Median size of largest lesion, cm | 8.7 (6.2-12.7) |
| (IQR) | |
| Median sum of largest diameters | 15.1 (8.3-14.9) |
| of lesions, cm (IQR) | |

AFP, ng/ml

| | |
|---|---|
| ≤400 ng/ml | 27 (82%) |
| >400 ng/ml | 6 (18%) |

Previous treatment

| | |
|---|---|
| No | 30 (91%) |
| Yes | 3 (9%) |
| Resection | 2 (6%) |
| Ablation | 1 (3%) |
| Median gross tumour volume, | 345.4 (161.2-824.1) |
| mL (IQR) | |
| Median planning target volume, | 474.9 (266.5-1113.5) |
| mL (IQR) | |
| Median prescribed dose, Gy‡ | 30.0 (30.0-35.0) |
| (IQR) | |

Data are n (%) unless otherwise specified.
BCLC = Barcelona Clinic Liver Cancer.
*At time of registration.
†Sum of the diameter of the largest tumor (cm) and the number of tumor greater than seven.
‡When multiple lesions were treated, some might have received a lower dose to meet the planning objectives.

TABLE 5
Listing of demographics and clinical characteristics of patients

| | | | Under- | | | | | | | | | Largest size | Sum of |
|---|---|---|---|---|---|---|---|---|---|---|---|---|---|
| | | | lying | Child- | | | | | Vascular | | No. of | ofliver | largest |
| | | | liver | Pugh | TNM | ALBI | | | invasion | AFP | liver | nodules | diameter of |
| Patient | Age | Sex | disease | score | Staging | (Grade) | ECOG | BOLC | (Yes/No) | (ng/ml) | nodules | (cm) | lesions (cm) |
| #1 | 77 | M | HCV | A5 | III | 1 | 0 | B | N | 61 | 3 | 5.7 | 15.2 |
| #2 | 68 | M | HBV | A5 | III | 1 | 0 | C | Y | 2.1 | 1 | 8.8 | 8.8 |
| #3 | 71 | M | Nil | A5 | III | 2 | 1 | C | Y | 66 | 1 | 13.2 | 13.2 |
| #4 | 57 | M | HBV | A5 | III | 1 | 1 | B | N | 4.8 | 3 | 7.9 | 15.9 |
| #5 | 75 | F | HBV | A6 | III | 2 | 1 | C | Y | 29.2 | 1 | 11.6 | 11.6 |
| #6 | 64 | M | HBV | A5 | III | 1 | 0 | C | Y | 40 | 2 | 5.7 | 9.3 |
| #7 | 56 | M | HBV | A5 | III | 1 | 0 | B | N | 21 | 2 | 6.8 | 8.1 |
| #8 | 77 | M | HBV | A5 | III | 1 | 1 | B | N | 4.7 | 2 | 5 | 7.1 |
| #9 | 76 | M | HBV | A5 | III | 1 | 1 | C | Y | 28 | 2 | 10.5 | 13.3 |
| #10 | 69 | M | HBV | B7 | I | 2 | 0 | A | N | 15 | 1 | 5.3 | 5.3 |
| #11 | 67 | M | Nil | A5 | III | 1 | 0 | C | Y | 4 | 1 | 8.7 | 8.7 |
| #12 | 77 | M | HBV | A5 | I | 1 | 0 | A | N | 623 | 1 | 8.7 | 8.7 |
| #13 | 59 | M | Nil | A5 | III | 1 | 0 | B | N | 14 | 1 | 11 | 11 |
| #14 | 75 | M | HBV | A5 | III | 1 | 0 | C | Y | 2751 | 1 | 14.5 | 14.5 |
| #15 | 59 | M | HBV | A5 | III | 1 | 0 | B | N | 3 | 2 | 10.2 | 17.4 |
| #16 | 64 | M | HCV | A5 | III | 1 | 0 | B | N | 69 | 2 | 11.5 | 12.8 |
| #17 | 51 | M | Nil | A6 | III | 2 | 0 | C | Y | 22238 | 1 | 17.5 | 17.5 |
| #18 | 66 | M | HBV | A6 | III | 2 | 0 | C | Y | 13.6 | 1 | 14.6 | 14.6 |
| #19 | 80 | M | HBV | A5 | III | 2 | 1 | C | Y | 1566 | 2 | 15.3 | 18.4 |
| #20 | 81 | M | HBV | A5 | III | 2 | 0 | C | Y | 8 | 1 | 7.4 | 7.4 |
| #21 | 72 | M | Nil | A6 | III | 2 | 0 | C | Y | 127620 | 3 | 15.1 | 31.1 |
| #22 | 73 | M | Nil | A6 | III | 2 | 1 | C | Y | 1 | 1 | 14.7 | 14.7 |
| #23 | 67 | M | HBV | A6 | I | 1 | 0 | A | N | 67496 | 1 | 7.6 | 7.6 |
| #24 | 56 | M | Nil | A6 | III | 1 | 0 | C | Y | 3 | 2 | 6.8 | 11.3 |
| #25 | 63 | M | HBV | A5 | III | 1 | 0 | B | N | 7 | 2 | 5 | 7.4 |
| #26 | 60 | M | HBV, HCV | A5 | III | 1 | 0 | C | Y | 6 | 2 | 7.2 | 8.7 |
| #27 | 70 | M | HBV | A5 | III | 1 | 0 | C | Y | 5 | 1 | 11.9 | 11.9 |
| #28 | 61 | M | HBV | A5 | III | 1 | 0 | C | Y | 7 | 2 | 6.7 | 9.1 |
| #29 | 74 | M | HBV | A6 | III | 2 | 0 | C | Y | 9 | 3 | 9.2 | 21.2 |
| #30 | 72 | M | HBV | A5 | I | 2 | 0 | A | N | 5 | 1 | 5.4 | 5.4 |
| #31 | 63 | M | HBV | A5 | III | 1 | 0 | C | Y | 24 | 2 | 6 | 6.5 |
| #32 | 70 | M | HBV | A5 | III | 1 | 0 | C | Y | 2 | 1 | 8.4 | 8.4 |
| #33 | 63 | M | HBV | A6 | III | 2 | 0 | C | Y | 4 | 1 | 18.3 | 18.3 |

Abbreviations:
HBV, Hepatitis B infected;
HCV, Hepatitis C infected;
ALBI, Albumin Bilirubin Score;
ECOG: Eastern Cooperative Group performance status;
AFP: Alpha-fetoprotein;
BCLC, Barcelona Clinical Liver Cancer Staging.

TABLE 6
Listing of resectability and tumor size before and after treatment

| | Reasons of | | Portal | Hepatic | | Baseline: | Post-treatment: | Best | | |
|---|---|---|---|---|---|---|---|---|---|---|
| | un [symbol] - | | vein | vein | | longest | longest | treatment: | Amendable | |
| | ability | BCLC | involve- | involve- | No of | diameter | diameter | response (Per | to curative | Surgery |
| Patient | HCC | stage | ment | ment | nodules | of lesion(s) | of lesion(s) | mRECIST) | surgery | done |
| #1 | BCLC B | B | No | No | 3 | 15.2 cm | 3.4 cm | PR | Yes, | Yes, RFA to |
| | and up-to- | | | | | (5.7 cm, | (3.4 cm, | | RFA | the residual |
| | seven-out | | | | | 4.3 cm, | 0 cm, | | | |
| | | | | | | 5.2 cm) | 0 cm) | | | |
| #2 | BCLC C | C | No | Middle | 1 | 8.8 cm | 6.1 cm | PR | No | No |
| #3 | BCLC C | C | No | Middle | 1 | 13.2 cm | 10.4 cm | SD | No | No |
| #4 | BCLC B | B | No | No | 3 | 15.9 cm | 17.2 cm | PD (New | No | No |
| | and up-to- | | | | | (7.9 cm, | (9.9 cm, | lesions) | | |
| | seven-out | | | | | 6.7 cm, | 6.5 cm, | | | |
| | | | | | | 1.3 cm) | 0.8 cm) | | | |
| #5 | BCLC C | C | No | Right, | 1 | 11.6 cm | 0 cm | CR | Yes, | No |
| | | | | middle, | | | | | resection | |
| | | | | left | | | | | | |
| #6 | BCLC C | C | No | Right, | 2 | 9.3 cm | 0 cm | CR | Yes, | No |
| | | | | middle | | (5.7 cm, | (0 cm, | | resection | |
| | | | | | | 3.6 cm) | 0 cm) | | | |
| #7 | BCLC B | B | No | No | 1 | 8.1 cm | 4.2 cm | PR | Yes, | Yes, Right |
| | and up-to- | | | | | (6. [symbol] cm, | (2.9 cm, | | resection | hepatectomy |
| | seven-out | | | | | 1.3 cm) | 1.3 cm) | | | |
| #8 | BCLC B | B | No | No | 2 | 7.1 cm | 0 cm | CR | Yes, RFA | No |
| | and up-to- | | | | | (5.0 cm, | (0 cm, | | | |
| | seven-out | | | | | 2.1 cm) | 0 cm) | | | |
| #9 | BCLC C | C | VP3 | Right, | 2 | 13.3 cm | 0 cm | CR | Yes, | No |
| | | | | middle | | (10.5 cm, | (0 cm, | | resection | |
| | | | | | | 2.8 cm) | 0 cm) | | | |
| #10 | Inadequate | A | No | No | 1 | 5.3 cm | 1 | PR | Yes, | Yes, RFA to |
| | liver | | | | | | | | RFA | the residual |
| | remnant | | | | | | | | | |
| | volume | | | | | | | | | |
| #11 | BCLC C | C | VP3 | Middle | 1 | 8.7 cm | 0 cm | CR | Yes, | No |
| | | | | | | | | | RFA | |
| #12 | Inadequate | A | No | No | 1 | 8.7 cm | 0 cm | CR | Yes, | No |
| | liver | | | | | | | | RFA | |
| | remanent | | | | | | | | | |
| | volume and | | | | | | | | | |
| | ICG > 15% | | | | | | | | | |
| #13 | BCLC B | B | No | No | 1 | 11 cm | 0 cm | CR | Yes, | No |
| | and up-to- | | | | | | | | resection | |
| | seven-out | | | | | | | | | |
| #14 | BCLC C | C | VPS | | 1 | 14.5 cm | 9.8 cm | PD (new | | No |
| | | | | | | | | lesions) | | |
| #15 | BCLC B | B | No | No | 2 | 17.4 cm | 5.9 cm | PR | Yes, | Yes, |
| | and up-to- | | | | | (10.2 cm, | (3.6 cm, | | resection | Right tri- |
| | seven-out | | | | | 7.2 cm) | 2.3 cm) | | | sectionectomy |
| #16 | BCLC B | B | No | No | 2 | 12.8 cm | 12.7 cm | SD | No | No |
| | and up-to- | | | | | (11.5 cm, | (11.4 cm, | | | |
| | seven-out | | | | | 1.3 cm) | 1.3 cm) | | | |
| #17 | BCLC C | C | No | Right, | 1 | 17.5 cm | 9.9 cm | PD (new | No | No |
| | | | | middle | | | | lesions) | | |
| #18 | BCLC C | C | VP3 | Right | 1 | 14.6 cm | 14.9 cm | PD (new | No | No |
| | | | | | | | | lesions) | | |
| #19 | BCLC C | C | VPS | Right, | 2 | 18.4 cm | 5.3 cm | PR | No | No |
| | | | | middle, | | (15.3 cm, | (3.6 cm, | | | |
| | | | | left | | 3.1 cm) | 1.7 cm) | | | |
| #20 | BCLC C | C | No | Right, | 1 | 7.4 cm | 0 cm | CR | Yes, | No |
| | | | | middle | | | | | RFA | |
| #21 | BCLC C | C | VP3 | Right, | 3 | 31.1 cm | 23.7 cm | PD (new | Na | No |
| | | | | middle | | (14 cm [symbol] | (11.8 cm, | lesions) | | |
| | | | | | | 15 cm, | 9.5 cm, | | | |
| | | | | | | 2 cm) | 2.4 cm) | | | |
| #22 | BCLC C | C | VP3 | Right, | 1 | 14.7 cm | 9.9 cm | PR | No | No |
| | | | | middle | | | | | | |
| #23 | Inadequate | A | No | No | 1 | 7.6 cm | 0 cm | CR | Yes, | No |
| | liver | | | | | | | | RFA | |
| | remnant | | | | | | | | | |
| | volume | | | | | | | | | |
| #24 | BCLC C | C | No | Right, | 2 | 11.3 cm | 0 cm | CR | Yes, | No |
| | | | | middle | | (6.8 cm, | (0 cm, | | LT | |
| | | | | | | 4.5 cm) | 0 cm) | | | |
| #25 | BCLC B | B | No | No | 2 | 7.4 cm | 0 cm | CR | Yes, | No |
| | and up-to- | | | | | (5 cm, | (0 cm, | | RFA | |
| | seven-out | | | | | 2.4 cm) | 0 cm) | | | |
| #26 | BCLC C | C | No | Right, | 2 | 8.7 cm | 1.5 cm | PD (new | No | No |
| | | | | middle | | (7.2 cm, | (0 cm, | lesions) | | |
| | | | | | | 1.5 cm) | 1.5 cm) | | | |
| #27 | BCLC C | C | No | Middle | 1 | 11.9 cm | 0 cm | CR | Yes, | No |
| | | | | | | | | | resection | |
| #28 | BCLC C | C | No | Middle, | 2 | 9.1 cm | 5.4 cm | PR | No | No |
| | | | | left | | (6.7 cm, | (3.6 cm, | | | |
| | | | | | | 2.4 cm) | 1.8 cm) | | | |
| #29 | BCLC C | C | No | Right, | 3 | 21.2 cm | 19 cm | SD | No | No |
| | | | | middle | | (9.2 cm, | (7.5 cm, | | | |
| | | | | | | 6.2 cm, | 6.1 cm, | | | |
| | | | | | | 5.8 cm) | 5.4 cm) | | | |
| #30 | Inadequate | A | No | No | 1 | 5.4 cm | 0 cm | CR | Yes, | No |
| | liver | | | | | | | | RFA | |
| | remnant | | | | | | | | | |
| | volume | | | | | | | | | |
| #31 | BCLC C | C | No | Middle | 2 | 26.5 cm | 0 cm | CR | Yes, | No |
| | | | | | | (5 cm, | (0 cm, | | resection | |
| | | | | | | 1.5 cm) | 0 cm) | | | |
| #32 | BCLC C | C | VP3 | No | 1 | 8.4 cm | 8 cm | PD | No | No |
| | | | | | | | | (new lesions) | | |
| #33 | BCLC C | C | No | Right | 1 | 18.3 cm | 17.6 cm | PD | No | No |
| | | | | | | | | (new lesions) | | |

Abbreviations:
BCLC, Barcelona Cancer Liver Cancer staging;
ICG: Indocyanine green;
VP3: Branched portal vein involvement;
mRECIST: modified RECIST;
CR, complete response;
PR, partial response;
SD, stable disease;
PD, progressive disease;
RFA, radiofrequency ablation;
LT, liver transplantation. [symbol]
indicates data missing or illegible when filed

At the time of data cutoff, all 33 patients had completed study treatment. Patients received a median of eight cycles (IQR 4.5-12.0) of avelumab. The median dose of stereotactic body radiotherapy was 30.0 Gy (range 30.0-35.0) in five fractions. 18 (55%) patients stopped study treatment because they were deemed amenable to treatment with curative intent. The remaining 15 (45%) patients were not deemed amenable to treatment with curative intent and discontinued study treatment for the following reasons: disease progression (n=10), adverse events (n=2), patient refusal (n=2), and death (n=1; FIG. 1). A summary of the stereotactic body radiotherapy dose parameters and subsequent treatment after disease progression are presented in the Table 7 and Table 8.

TABLE 7
Summary of dosimetric characteristics of stereotactic body radiotherapy

| | N = 33 (%) |
|---|---|
| GTV volume, ml, median (interquartile range) | 190.2 (472.6) |
| PTV volume, ml, median (interquartile range) | 276.4 (565) |
| Liver-GTV volume, ml, median (interquartile range) | 1108.1 (400.6) |
| Prescribed dose, Gy, median (interquartile range) | 30 (5) |
| Liver mean dose, Gy, median (interquartile range) | 15.0 (2.2) |
| V15 Gy, ml, median (interquartile range) | 707.6 (301.2) |
| Veff, %, median (interquartile range) | 0.28 (0.07) |
| Physical NTCP, %, median (interquartile range) | 10.28 (18.53) |
| Biological NTCP, %, median (interquartile range) | 0.54 (0.99) |

Abbreviations:
GTV, gross tumor volume;
PTV, planning target volume;
V15, volume of liver received >15 Gy;
Veff, effective liver volume;
NTCP, normal tissue complication probability.

TABLE 8
Pattern of post-progression therapy

| | N = 33 (%) |
|---|---|
| Total number of patients with at least one | 7 (21) |
| post-treatment systemic therapy | |

Number of lines of post-treatment systemic therapy

| One line | 4 (12) |
|---|---|
| Two lines | 1 (3) |
| Three lines or more | 2 (6) |
| Regimen | |
| Lenvatinib | 6 (18) |
| Sorafenib | 3 (9) |
| Regorafenib | 1 (3) |
| Cabozatinib | 1 (3) |
| Atezolizumab-Bevacizumab | 1 (3) |
| Immunotherapy | 1 (3) |
| Total number of patients with at least | |
| one post-treatment loco-regional therapy | |
| TACE | 9 (27) |

Abbreviation:
TACE, transarterial chemoembolization

Of the 18 (55%) of 33 patients who were deemed amenable to curative treatment after receiving the START-FIT regimen, eight were deemed amenable to resection, nine were deemed amenable to radiofrequency ablation, and one was deemed amenable to liver transplantation (Table 6). Among all 33 patients, 14 (42%) had a complete response and four (12%) had curative treatment (two patients had resection and two patients had radiofrequency ablation). Pathological review of the tumor specimens from the two patients who underwent resection showed a tumor necrosis rate that was 50% or higher. All patients who had a compete response opted for active surveillance (FIG. 1).

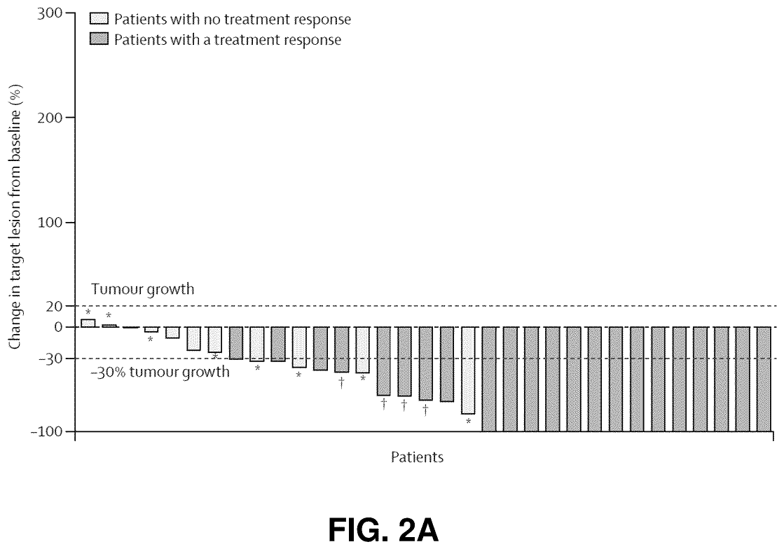

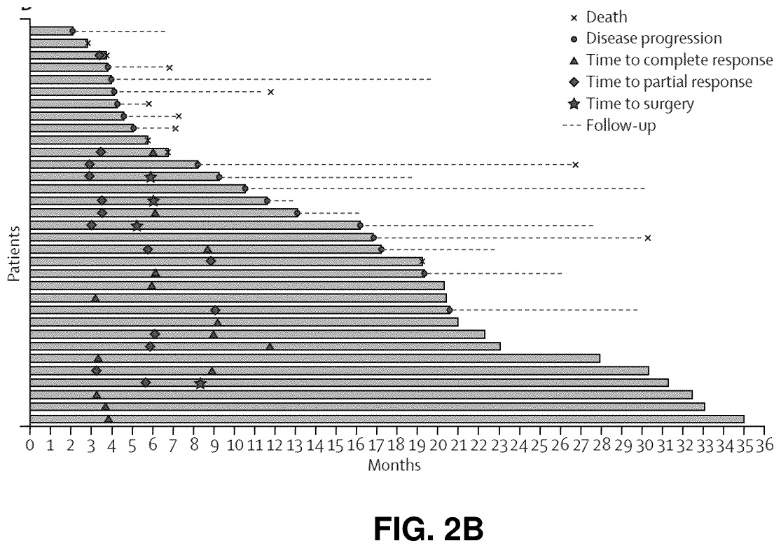

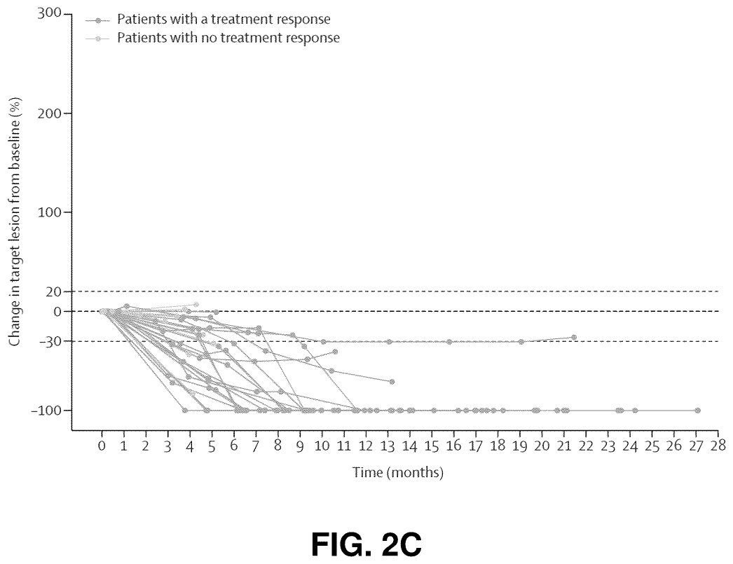

The confirmed objective response rate was 67% (95% CI 48-82), according to investigator review. The compete response rate was 42% (26-61), and the partial response rate was 24% (11-42; Table 9). Three (9%) patients had stable disease, and eight (24%) patients had progressive disease (Table 9). The disease control rate was 70% (95% CI 51-84; Table 9). According to independent review, the objective response rate was 67% (48-82) and the disease control rate was 73% (55-87; Table 9). 31 (94%) of 33 patients had target lesions that showed tumor regression (FIG. 2A). Tumor responses as determined by RECIST (version 1.1) and immune RECIST are shown in the Table 10.

TABLE 9
Confirmed antitumor activity, evaluated by modified RECIST

| | Per investigator | Per independent |
|---|---|---|
| | review | review |
| Objective response rate* | 22 (67%, 48-82) | 22 (67%, 48-82) |
| Complete response | 14 (42%, 26-61) | 14 (42%, 26-61) |
| Partial response | 8 (24%, 11-42) | 8 (24%, 11-42) |
| Suitable for treatment | 4 (12%, 3-28) | 4 (12%, 3-28) |
| with curative intent | | |
| Unsuitable for treatment | 4 (12%, 3-28) | 4 (12%, 3-28) |
| with curative intent | | |
| Stable disease | 3 (9%, 2-24) | 5 (15%, 5-32) |
| Progressive disease | 8 (24%, 11-42) | 6 (18%, 7-36) |
| Disease control rate† | 23 (70%, 51-84) | 24 (73%, 55-87) |
| Median duration of | 20.2 (11.2-21.7) | 20.2 (11.2-21.7) |
| response, months (IQR) | | |

Local control rate, % (95% CI)‡

| | | |
|---|---|---|
| 6 months | 98% (94-100) | 98% (94-100) |
| 12 months | 92% (84-100) | 92% (84-100) |
| 24 months | 92% (84-100) | 92% (84-100) |

Data are n (%, 95% CI) of 33 patients, unless otherwise stated.
RECIST = Response Evaluation Criteria in Solid Tumors.
*Complete response or partial response.
†Complete response, partial response, or stable disease for at least 6 months.
‡n = 55 lesions.

TABLE 10
Objective response rates, median progression free survival and overall survival
determined by RECIST, mRECIST, and iRECIST

| Response | | | | | | | | | |
|---|---|---|---|---|---|---|---|---|---|
| Evaluation | RECIST 1.1 | PFS | OS | mRECIST | PFS | OS | iRECIST | PFS | OS |
| ORR (CR + PR). | [symbol] 24 | NR | NR | 22 [symbol] 67 | 26 [symbol] 7 | NR | 8 [symbol] 24 | NR | NR |
| No [symbol] % | (11-42) | | | (48-82) | (21.0-32.5) | | (11-42) | | |
| (95% CI) | | | | | | | | | |
| CR, No [symbol] % | 0 [symbol] 0 | NA | NA | 14 [symbol] 42 | NR | NR | 0 [symbol] 0 | NA | NA |
| (95% CI) | (0-0) | | | (26-61) | | | (0-0) | | |
| PR. No [symbol] % | 8 [symbol] 24 | NR | NR | 8 [symbol] 24 | 20.2 | NR | 8 [symbol] 24 | NR | NR |
| (95% CI) | (11-42) | | | (11-42) | (13.1-27.3) | | (11-42) | | |
| SD. No [symbol] % | 17 [symbol] 52 | 23 [symbol] 0 | NR | 3 [symbol] 9 | NR | NR | 17 [symbol] 52 | 23.0 | NR |
| (95% CI) | (34-69) | (19.5-26.5) | | (2-24) | | | (34-69) | (19.5-26.5) | |
| PD. No [symbol] 9% | [symbol] 24 | 5.1 | 7.3 | 8 [symbol] 24 | [symbol] 1 | 7.3 | 8 [symbol] 24 | [symbol] 1 | 7.3 |
| (95% CI) | (11-42) | (3.4-6.7) | (1.7-12.8) | (11-42) | (3-4-6.7) | (1.7-12. [symbol] ) | (11-42) | (3.4-6.7) | (1.7-12.8) |
| P value | | 0.18 | 0.23 | | 0.001 | 0-012 | | 0.18 | 0.23 |
| DCR | 22 [symbol] 69 | | | 23 [symbol] 72 | | | 22.69 | | |
| (CR + PR + SD ≥ | (50-84) | | | (55-84) | | | (50-84) | | |
| 6 months) | | | | | | | | | |
| No [symbol] % | | | | | | | | | |
| (95% CI) | | | | | | | | | |
| Median DOR, | 21.2 | | | 20.2 | | | 21.2 | | |
| months (range) | (2.3-28.9) | | | (0.2-28.9) | | | (2.3-28.9) | | |
| Median PFS, | 20.7 | | | 20.7 | | | 20.7 | | |
| months (range) | (14.6-26 [symbol] 8) | | | (14.6-26 [symbol] ) | | | (14.6-26 [symbol] ) | | |

Abbreviations:
ORR, objective response rate;
CR, complete response;
PR, partial response;
SD, stable disease;
PD, progressive disease;
DCR, disease control rate;
DOR, duration of response;
PFS: progression free survival;
OS: overall survival;
NR: not reached. [symbol]
indicates data missing or illegible when filed

The median time to treatment response was 3.8 months (95% CI 2.4-8.7). The median duration of response was 20.2 months (IQR 11.2-21.7). Notably, all 14 patients who had a complete response had stopped avelumab upon achieving a complete response. The median number of cycles of avelumab received among patients who had a complete response was nine (IQR 6.8-12.3). 11 (79%) of 14 patients who had a complete response did not have disease progression at the time of data cutoff after a median follow-up of 17.2 months (IQR 7.8-25.8; range 6.8-28.1; FIG. 2B). Changes in target lesions over time are presented in FIG. 2C. None of the patients relapsed within the radiation field (FIGS. 2B-2C).

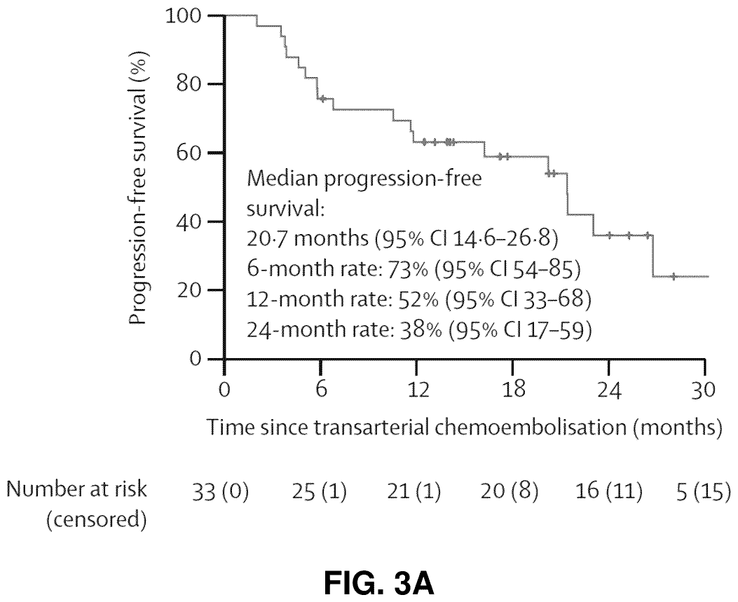

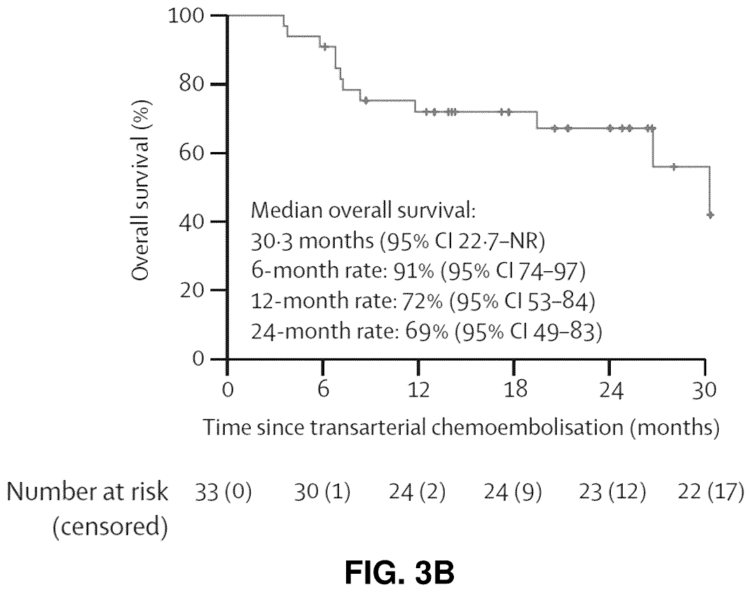

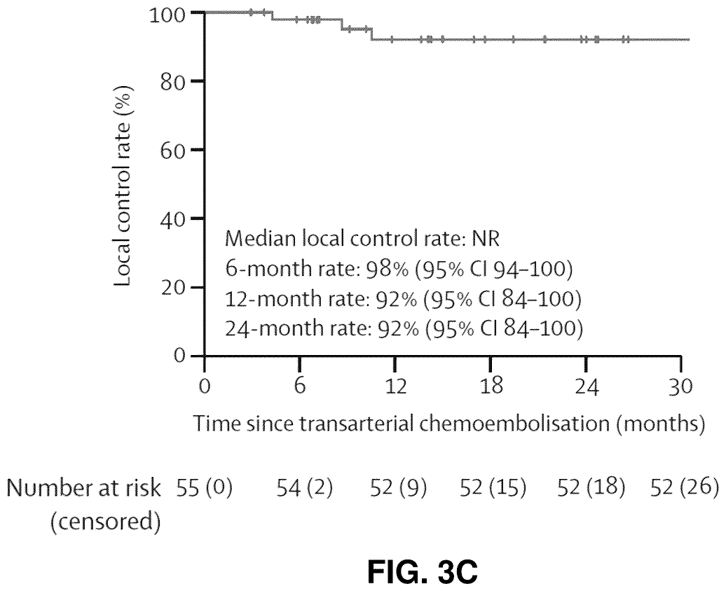

After a median follow-up of 17.2 months (IQR 7.8-25.8; range 3.5-31.6) for the entire cohort, median progression-free survival was 20.7 months (95% CI 14.6-26.8), median time to progression was 21.4 months (16.4-26.4), and median overall survival was 30.3 months (22.7 to not reached; FIGS. 3A-3B). The 12-month local control rate (n=55 lesions) was 92% (95% CI 84-100) and the 24-month local control rate (n=55 lesions) was 92% (84-100; Table 9; FIG. 3C). The 12-month progression-free survival rate was 52% (95% CI 33-68) and the 24-month progression-free survival rate was 38% (18-59; FIG. 3A). The 12-month overall survival rate was 72% (95% CI 53-84) and the 24-month overall survival rate was 69% (49-83; FIG. 3B). The 24-month overall survival rate of patients amenable to surgery (n=18) was 94% (79-100). Among them, the 24-month overall survival rate of patients who had a complete response and opted for close surveillance (n=14) was 92% (78.5-100.0) and the 24-month overall survival of patients who underwent curative treatment (n=4) was 100% (94.2-100.0). The causes of death among the 12 patient events recorded included disease progression (n=6), decompensated liver failure (n=2), medical illness (n=2), suicide (n=1), and accident (n=1; FIG. 1). Of the 17 patients who eventually developed disease progression, 13 (77%) presented with intrahepatic out-of-field progression Table 11.

TABLE 11
Pattern of disease progression

| | | | Site of first |
|---|---|---|---|
| | 6 months | 12 months | progression |
| | N (%) | N (%) | N (%) |
| Evaluable patients | 33 | 23 | 33 |
| No progression | 25 (76%) | 17 (74%) | 16 (48%) |
| Progression | 8 (24%) | 6 (26%) | 17 (52%) |
| In-field progression | 0 (0%) | 1 (17%) | 1 (6%) |
| New intra-hepatic out-field | 6 (75%) | 4 (67%) | 13 (77%) |
| lesion | | | |
| New extra-hepatic lesion | 1 (13%) | 0 (0%) | 2 (12%) |
| New vascular invasion | 0 (0%) | 0 (0%) | 0 (0%) |
| In-field progression + | 1 (13%) | 0 (0%) | 1 (6%) |
| new intra-hepatic out-field | | | |
| progression | | | |
| In-field progression + | 0 (0%) | 1 (17%) | 0 (0%) |
| new intra-hepatic out-field | | | |
| progression + new extra- | | | |
| hepatic lesion | | | |

Treatment-related adverse events occurred in all patients during the study treatment period (Table 12). 11 (33%) of 33 patients had at least one treatment-related adverse event that was grade 3 or worse. The most common treatment-related adverse events that were grade 3 or worse were increased alanine aminotransferase or aspartate aminotransferase (five [15%] of 33 patients; Table 12), increased bilirubin (two [6%]), and an increase in both alanine aminotransferase or aspartate aminotransferase and bilirubin (two [6%]) after TACE. All seven (21%) patients with treatment-related hepatic impairment recovered uneventfully and were managed conservatively. The most common immune-related adverse events of grade 3 or worse, which occurred in five (15%) of 33 patients, were hepatitis and dermatitis (Table 12). All patients with immune-related adverse events responded to steroid treatment (oral prednisolone 1 mg/kg). However, treatment with avelumab was permanently discontinued in two patients due to immune-related adverse events. Temporary dose interruption of avelumab occurred in seven (21%) patients with a median duration of interruption of 4 weeks (IQR 2-5; range 1-8).

TABLE 12
Treatment-related adverse events

| | Grade 1-2 | Grade 3 | Grade 4 | Grade 5 |
|---|---|---|---|---|
| Any treatment-related adverse | 27 (82%) | 3 (9%) | 8 (24%) | 0 |
| events | | | | |
| Lead to discontinuation | 0 | 1 (3%) | 1 (3%) | 0 |

Constitutional

| | | | | |
|---|---|---|---|---|
| Fatigue | 21 (64%) | 0 | 0 | 0 |
| Fever | 10 (30%) | 0 | 0 | 0 |
| Infusion-related reaction | 7 (21%) | 0 | 0 | 0 |
| Weight decreased | 7 (21%) | 0 | 0 | 0 |
| Ascites | 5 (15%) | 1 (3%) | 0 | 0 |
| Weight gained; dizziness; | 11 (33%) | 1 (3%) | 1 (3%) | 0 |
| hoarseness | | | | |

Skin

| | | | | |
|---|---|---|---|---|
| Rash, maculopapular | 10 (30%) | 1 (3%) | 0 | 0 |
| Rash, acneiform | 3 (9%) | 0 | 0 | 0 |
| Rash, urticarial | 3 (9%) | 1 (3%) | 0 | 0 |
| Herpes zoster | 3 (9%) | 0 | 0 | 0 |
| Pruritus | 8 (24%) | 1 (3%) | 0 | 0 |
| Dry skin | 11 (33%) | 1 (3%) | 0 | 0 |
| Bilateral lower extremity oedema | 1 (3%) | 0 | 0 | 0 |

Gastrointestinal

| | | | | |
|---|---|---|---|---|
| Decreased appetite | 9 (27%) | 0 | 0 | 0 |
| Nausea | 14 (42%) | 1 (3%) | 0 | 0 |
| Vomiting | 7 (21%) | 0 | 0 | 0 |
| Diarrhoea | 4 (12%) | 0 | 0 | 0 |
| Stomatitis | 4 (12%) | 0 | 0 | 0 |
| Constipation | 5 (15%) | 0 | 0 | 0 |
| Gastritis or gastrointestinal bleed | 1 (3%) | 0 | 0 | 0 |
| Others | 6 (18%) | 0 | 0 | 0 |

Hepatic

| | | | | |
|---|---|---|---|---|
| Abdominal pain | 7 (21%) | 0 | 0 | 0 |
| Alanine aminotransferase | 13 (39%) | 5 (15%) | 1 (3%) | 0 |
| increased | | | | |
| Aspartate aminotransferase | 16 (48%) | 1 (3%) | 6 (18%) | 0 |
| increased | | | | |
| Gamma-glutamyl transferase | 2 (6%) | 1 (3%) | 0 | 0 |
| increased | | | | |
| Bilirubin increased | 24 (73%) | 2 (6%) | 0 | 0 |
| Alkaline phosphatase increased | 19 (58%) | 1 (3%) | 0 | 0 |

Laboratory

| | | | | |
|---|---|---|---|---|
| Anaemia | 19 (58%) | 0 | 0 | 0 |
| Leukopenia | 14 (42%) | 0 | 0 | 0 |
| Neutropenia | 2 (6%) | 0 | 0 | 0 |
| Thrombocytopenia | 10 (30%) | 0 | 0 | 0 |
| Hyperglycaemia | 1 (3%) | 0 | 0 | 0 |
| Hyponatraemia | 13 (39%) | 0 | 1 (3%) | 0 |
| Hypokalaemia | 7 (21%) | 0 | 0 | 0 |
| Hypercreatinine | 3 (9%) | 0 | 0 | 0 |
| Hypothyroidism | 5 (15%) | 0 | 0 | 0 |

Immune-related

| | | | | |
|---|---|---|---|---|
| Hepatitis | 2 (6%) | 3 (9%) | 0 | 0 |
| Dermatitis | 2 (6%) | 2 (6%) | 0 | 0 |
| Other immune-related events* | 5 (15%) | 0 | 0 | 0 |

Data are n (%) of 33 patients.
*Hypothyroidism; adrenal insufficiency.

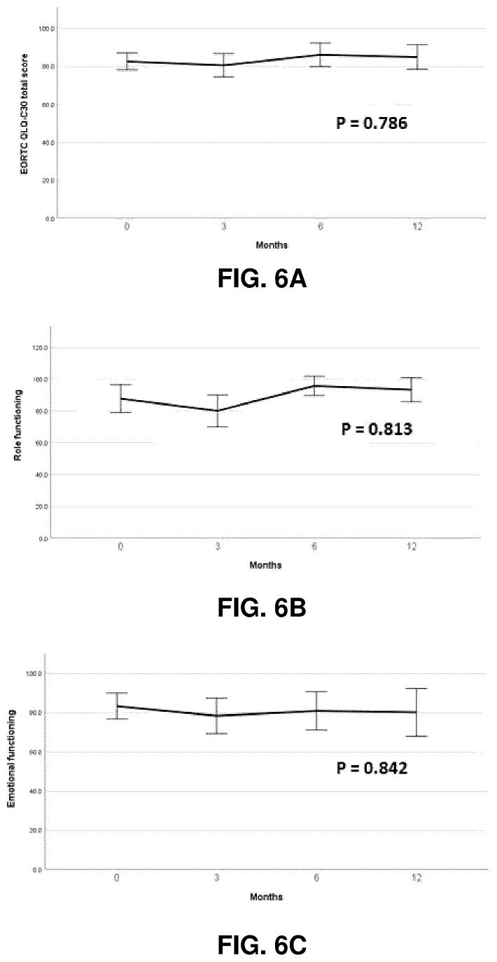

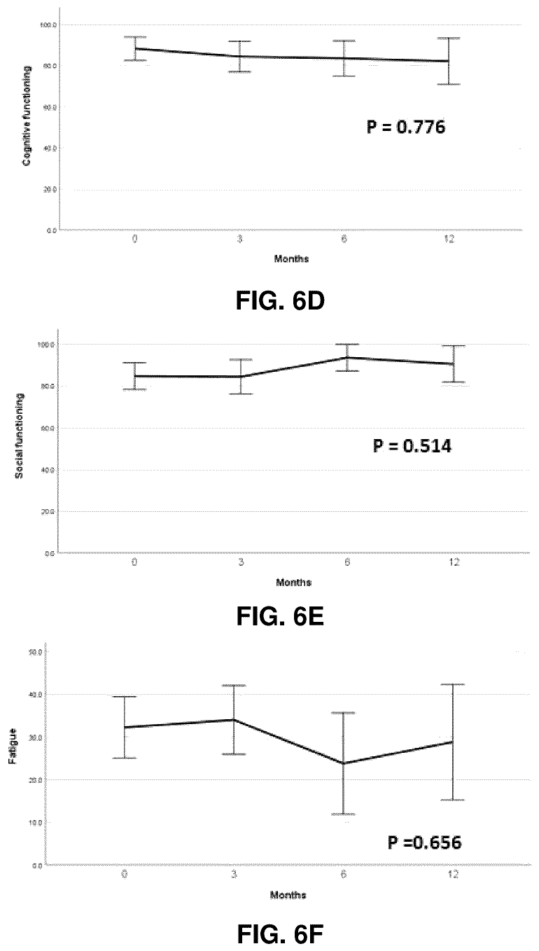

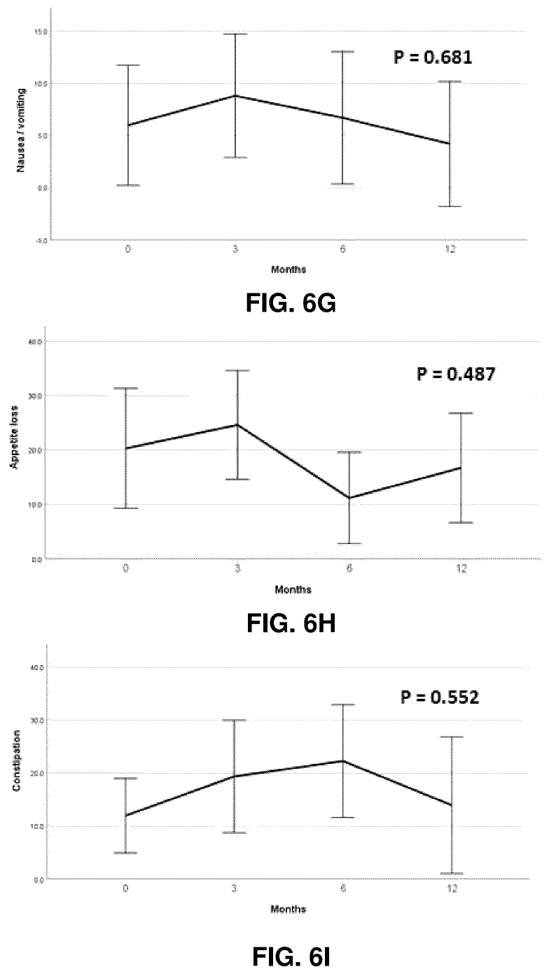

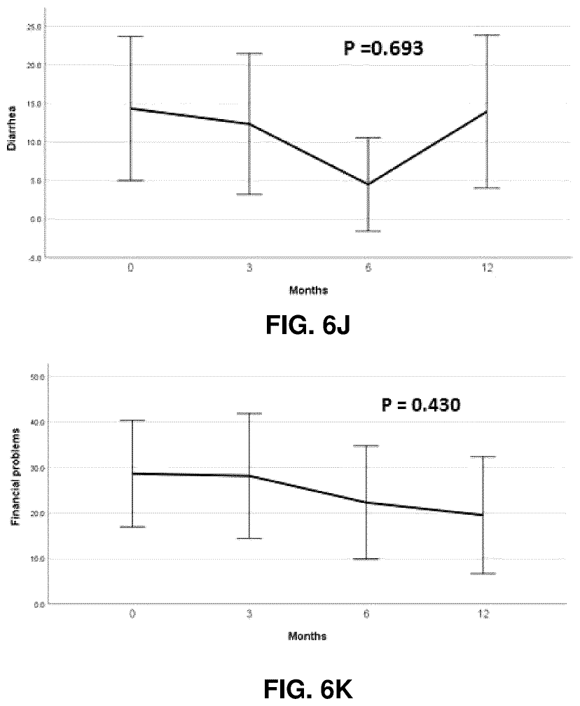

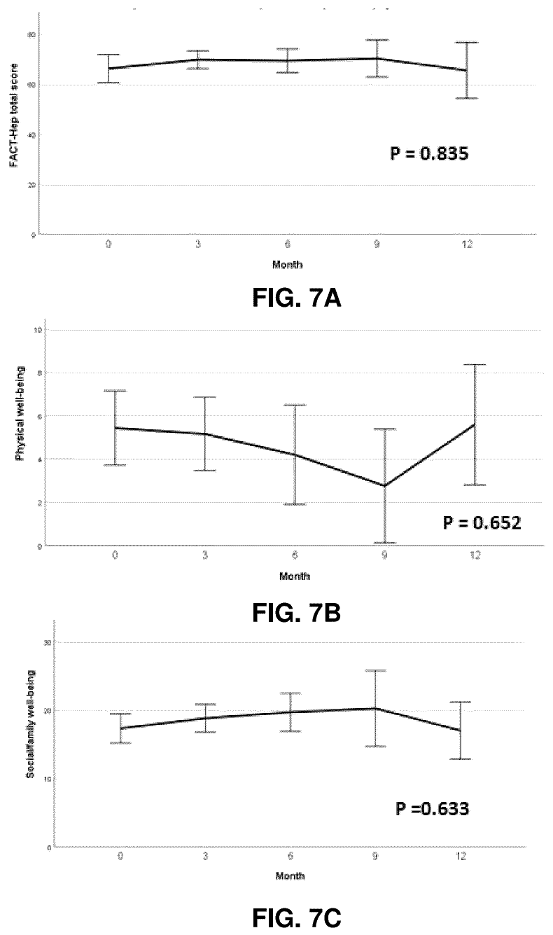

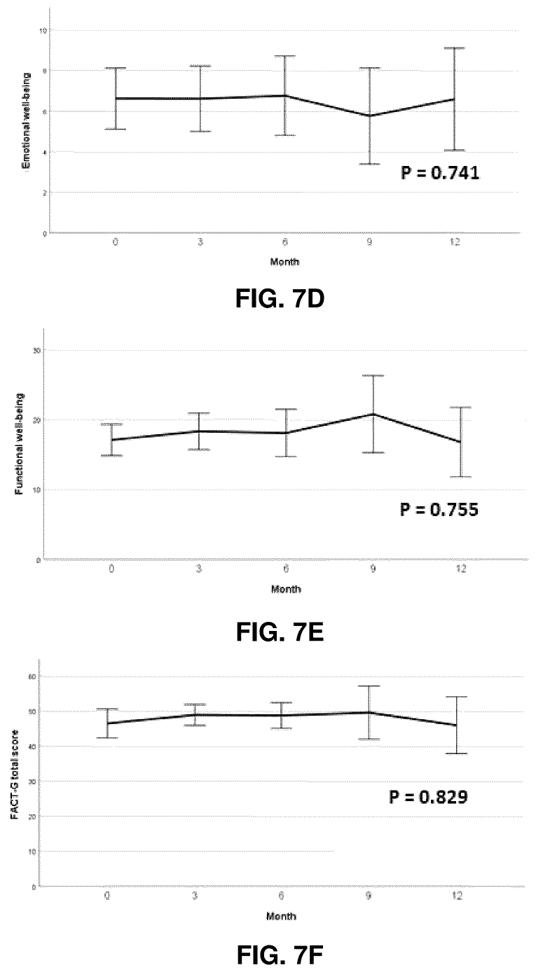

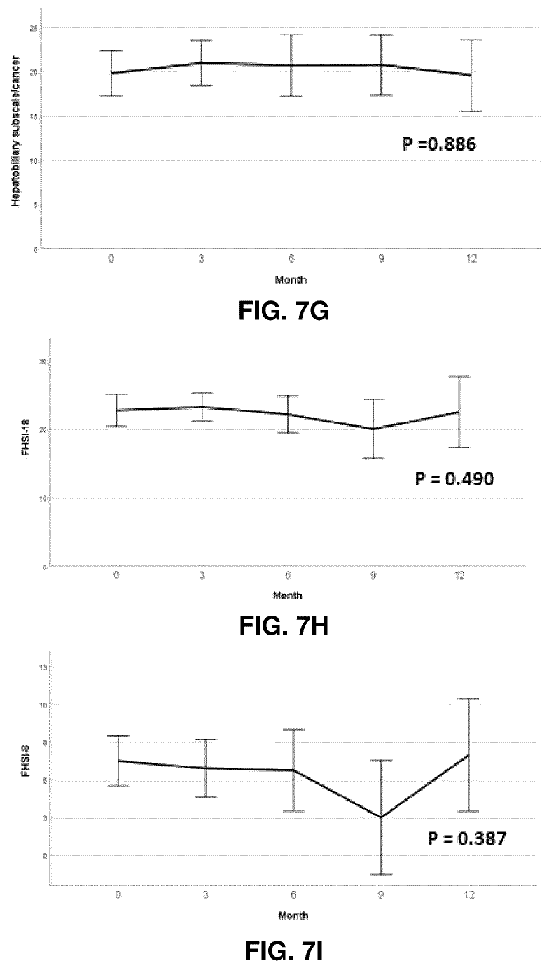

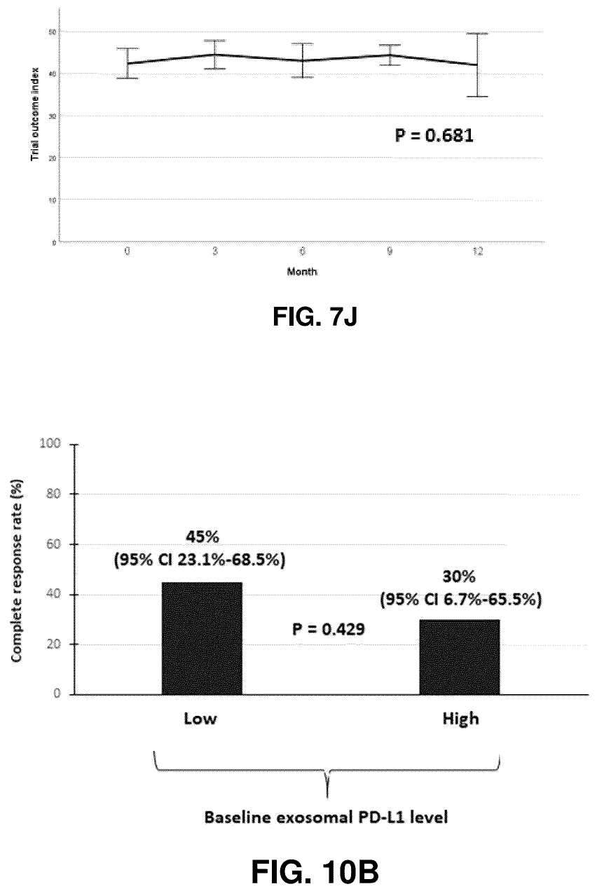

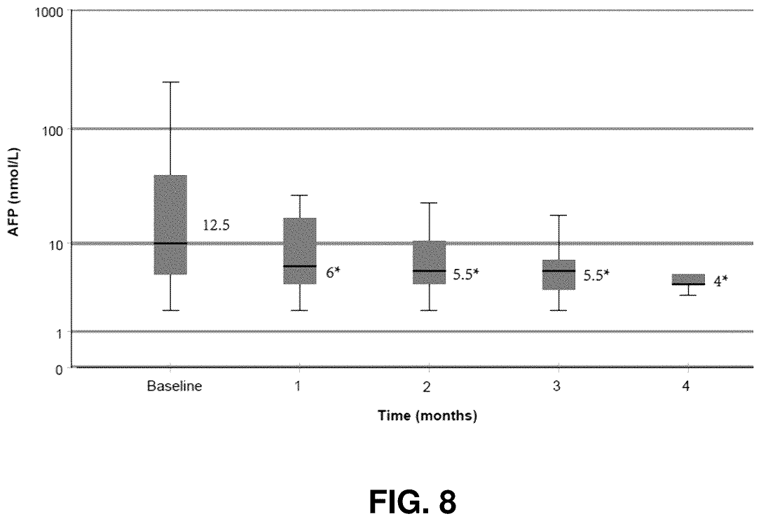

Treatment-related adverse events related to TACE, stereotactic body radiotherapy, and avelumab are presented separately in the Table 13, Table 14, and Table 15. No radiation-induced liver disease or treatment-related deaths were observed. Child-Pugh score deterioration of at least 2 points was detected in three (12%) of 25 patients at 3 months, in three (14%) of 22 patients at 6 months, and in one (6%) of 17 patients at 12 months after enrolment. There was no significant deterioration in quality-of-life scores throughout the study period (FIGS. 6A-6K and FIGS. 7A-7J) Decreasing serum AFP concentrations were observed in most patients (28 [84.4%] of 33) enrolled in the study (FIG. 8). However, there was no correlation between serum AFP changes over time and radiological responses. Median progression-free survival was 22.2 months (95% CI 18.4-27.6) in the 21 patients with albumin-bilirubin grade 1 and 6.8 months (95% CI 0-14.8) in the 12 patients with albumin-bilirubin grade 2 (p=0.070).

TABLE 13
TACE related adverse event

| | Grade 1-2 | Grade 3 | Grade 4 | Grade 5 |
|---|---|---|---|---|
| Any* | 21 (64%) | 0 (0%) | 7 (21%) | 0 (0%) |
| Lead to | 0 (0%) | 0 (0%) | 0 (0%) | 0 (0%) |
| discontinuation | | | | |

Adverse events
Constitutional

| | | | | |
|---|---|---|---|---|
| Fatigue | 8 (24%) | 0 (0%) | 0 (0%) | 0 (0%) |
| Fever | 9 (27%) | 0 (0%) | 0 (0%) | 0 (0%) |
| Infusion-related | 0 (0%) | 0 (0%) | 0 (0%) | 0 (0%) |
| reaction | | | | |
| Musculoskeletal | 0 (0%) | 0 (0%) | 0 (0%) | 0 (0%) |
| pain | | | | |
| Edema | 0 (0%) | 0 (0%) | 0 (0%) | 0 (0%) |
| Weight | 0 (0%) | 0 (0%) | 0 (0%) | 0 (0%) |
| decreased | | | | |
| ascites | 2 (6%) | 0 (0%) | 0 (0%) | 0 (0%) |
| Others | 3 (9%) | 0 (0%) | 1 (3%) | 0 (0%) |

Gastrointestinal

| | | | | |
|---|---|---|---|---|
| Decreased | 5 (15%) | 0 (0%) | 0 (0%) | 0 (0%) |
| appetite | | | | |
| Nausea | 8 (24%) | 0 (0%) | 0 (0%) | 0 (0%) |
| Vomiting | 2 (6%) | 0 (0%) | 0 (0%) | 0 (0%) |
| Diarrhea | 0 (0%) | 0 (0%) | 0 (0%) | 0 (0%) |
| Stomatitis | 0 (0%) | 0 (0%) | 0 (0%) | 0 (0%) |
| Constipation | 1 (3%) | 0 (0%) | 0 (0%) | 0 (0%) |
| Gastritis/GI bleed | 0 (0%) | 0 (0%) | 0 (0%) | 0 (0%) |
| Others | 4 (12%) | 0 (0%) | 0 (0%) | 0 (0%) |

Hepatic

| | | | | |
|---|---|---|---|---|
| Abdominal pain | 6 (18%) | 0 (0%) | 0 (0%) | 0 (0%) |
| ALT increased | 8 (24%) | 4 (12%) | 1 (3%) | 0 (0%) |
| AST increased | 13 (39%) | 0 (0%) | 5 (15%) | 0 (0%) |
| GGT increased | 0 (0%) | 1 (3%) | 0 (0%) | 0 (0%) |
| Bilirubin raised | 21 (64%) | 2 (6%) | 0 (0%) | 0 (0%) |
| ALP increased | 11 (33%) | 1 (3%) | 0 (0%) | 0 (0%) |

Laboratory

| | | | | |
|---|---|---|---|---|
| Anemia | 12 (36%) | 0 (0%) | 0 (0%) | 0 (0%) |
| Leukopenia | 1 (3%) | 0 (0%) | 0 (0%) | 0 (0%) |
| Neutropenia | 0 (0%) | 0 (0%) | 0 (0%) | 0 (0%) |
| Thrombocytopenia | 2 (6%) | 0 (0%) | 0 (0%) | 0 (0%) |
| Hypothyroidism | 0 (0%) | 0 (0%) | 0 (0%) | 0 (0%) |
| Amylase increased | 0 (0%) | 0 (0%) | 0 (0%) | 0 (0%) |
| Lipase increased | 0 (0%) | 0 (0%) | 0 (0%) | 0 (0%) |
| Hyperglycemia | 0 (0%) | 0 (0%) | 0 (0%) | 0 (0%) |
| Hyponatremia | 8 (24%) | 0 (0%) | 1 (3%) | 0 (0%) |
| Hypokalemia | 3 (9%) | 0 (0%) | 0 (0%) | 0 (0%) |
| Hypercreatinine | 0 (0%) | 0 (0%) | 0 (0%) | 0 (0%) |

Abbreviations:
GI, gastrointestinal;
ALT: Alanine transaminase;
AST: Aspartate transaminase;
GGT: Gamma-glutamyl Transferase.
*Data are number of patients with the worst toxicity grade

TABLE 14
SBRT related adverse event

| | Grade 1-2 | Grade 3 | Grade 4 | Grade 5 |
|---|---|---|---|---|
| Any* | 25 (76%) | 0 (0%) | 0 (0%) | 0 (0%) |
| Lead to | 0 (0%) | 0 (0%) | 0 (0%) | 0 (0%) |
| discontinuation | | | | |
| Adverse events | | | | |
| Constitutional | | | | |
| Fatigue | 11 (33%) | 0 (0%) | 0 (0%) | 0 (0%) |
| Fever | 0 (0%) | 0 (0%) | 0 (0%) | 0 (0%) |
| Infusion-related | 0 (0%) | 0 (0%) | 0 (0%) | 0 (0%) |
| reaction | | | | |
| Musculoskeletal | 0 (0%) | 0 (0%) | 0 (0%) | 0 (0%) |
| pain | | | | |
| Edema | 0 (0%) | 0 (0%) | 0 (0%) | 0 (0%) |
| Weight | 0 (0%) | 0 (0%) | 0 (0%) | 0 (0%) |
| decreased | | | | |
| Ascites | 2 (6%) | 0 (0%) | 0 (0%) | 0 (0%) |
| Others | 0 (0%) | 0 (0%) | 0 (0%) | 0 (0%) |

Gastrointestinal

| | | | | |
|---|---|---|---|---|
| Decreased | (3%) | 0 (0%) | 0 (0%) | 0 (0%) |
| appetite | | | | |
| Nausea | 4 (12%) | 0 (0%) | 0 (0%) | 0 (0%) |
| Vomiting | 2 (6%) | 0 (0%) | 0 (0%) | 0 (0%) |
| Diarrhea | 4 (12%) | 0 (0%) | 0 (0%) | 0 (0%) |
| Stomatitis | 0 (0%) | 0 (0%) | 0 (0%) | 0 (0%) |
| Constipation | 0 (0%) | 0 (0%) | 0 (0%) | 0 (0%) |
| Gastritis/GI bleed | 0 (0%) | 0 (0%) | 0 (0%) | 0 (0%) |
| Others | 1 (3%) | 0 (0%) | 0 (0%) | 0 (0%) |

Hepatic

| | | | | |
|---|---|---|---|---|
| Abdominal pain | 1 (3%) | 0 (0%) | 0 (0%) | 0 (0%) |
| ALT increased | 1 (3%) | 0 (0%) | 0 (0%) | 0 (0%) |
| AST increased | 4 (12%) | 0 (0%) | 0 (0%) | 0 (0%) |
| GGT increased | 0 (0%) | 0 (0%) | 0 (0%) | 0 (0%) |
| Bilirubin raised | 3 (9%) | 0 (0%) | 0 (0%) | 0 (0%) |

Laboratory

| | | | | |
|---|---|---|---|---|
| Anemia | 6 (18%) | 0 (0%) | 0 (0%) | 0 (0%) |
| Leukopenia | 9 (27%) | 0 (0%) | 0 (0%) | 0 (0%) |
| Neutropenia | 1 (3%) | 0 (0%) | 0 (0%) | 0 (0%) |
| Thrombocytopenia | 7 (21%) | 0 (0%) | 0 (0%) | 0 (0%) |
| Hypothyroidism | 0 (0%) | 0 (0%) | 0 (0%) | 0 (0%) |
| Amylase increased | 0 (0%) | 0 (0%) | 0 (0%) | 0 (0%) |
| Lipase increased | 0 (0%) | 0 (0%) | 0 (0%) | 0 (0%) |
| Hyperglycemia | 0 (0%) | 0 (0%) | 0 (0%) | 0 (0%) |
| Hyponatremia | 2 (6%) | 0 (0%) | 0 (0%) | 0 (0%) |
| Hypokalemia | 0 (0%) | 0 (0%) | 0 (0%) | 0 (0%) |
| Hypercreatinine | 0 (0%) | 0 (0%) | 0 (0%) | 0 (0%) |
| Hypothyroidism | 0 (0%) | 0 (0%) | 0 (0%) | 0 (0%) |

Abbreviations:
GI, gastrointestinal;
ALT: Alanine transaminase;
AST: Aspartate transaminase;
GGT: Gamma-glutamyl Transferase
*Data are number of patients with the worst toxicity grade

TABLE 15
Avelumab related adverse event

| | | | | |
|---|---|---|---|---|
| Any* | 27 (82%) | 4 (12%) | 1 (3%) | 0 (0%) |
| Lead to | 0 (0%) | 1 (3%) | 1 (3%) | 0 (0%) |
| discontinuation | | | | |

Adverse events
Constitutional

| | | | | |
|---|---|---|---|---|
| Fatigue | 9 (27%) | 0 (0%) | 0 (0%) | 0 (0%) |
| Fever | 1 (3%) | 0 (0%) | 0 (0%) | 0 (0%) |
| Infusion-related | 7 (21%) | 0 (0%) | 0 (0%) | 0 (0%) |
| reaction | | | | |
| Musculoskeletal | 0 (0%) | 0 (0%) | 0 (0%) | 0 (0%) |
| pain | | | | |
| Edema | 0 (0%) | 0 (0%) | 0 (0%) | 0 (0%) |
| Weight | 7 (21%) | 0 (0%) | 0 (0%) | 0 (0%) |
| decreased | | | | |
| Others | 9 (27%) | 0 (0%) | 0 (0%) | 0 (0%) |

Skin

| | | | | |
|---|---|---|---|---|
| Rash, | 6 (9%) | 1 (3%) | 0 (0%) | 0 (0%) |
| maculopapular | | | | |
| Rash, acneiform | 0 (0%) | 0 (0%) | 0 (0%) | 0 (0%) |
| Rash, urticarial | 2 (6%) | 1 (3%) | 0 (0%) | 0 (0%) |
| Herpes zoster | 2 (6%) | 0 (0%) | 0 (0%) | 0 (0%) |
| Pruritus | 8 (24%) | 1 (3%) | 0 (0%) | 0 (0%) |
| Dry skin | 6 (9%) | 0 (0%) | 0 (0%) | 0 (0%) |
| Others | 0 (0%) | 0 (0%) | 0 (0%) | 0 (0%) |

Gastrointestinal

| | | | | |
|---|---|---|---|---|
| Decreased | 4 (12%) | 0 (0%) | 0 (0%) | 0 (0%) |
| appetite | | | | |
| Nausea | 2 (6%) | 0 (0%) | 0 (0%) | 0 (0%) |
| Vomiting | 1 (3%) | 0 (0%) | 0 (0%) | 0 (0%) |
| Diarrhea | 4 (12%) | 0 (0%) | 0 (0%) | 0 (0%) |
| Stomatitis | 0 (0%) | 0 (0%) | 0 (0%) | 0 (0%) |
| Constipation | 2 (6%) | 0 (0%) | 0 (0%) | 0 (0%) |
| Gastritis/GI bleed | 0 (0%) | 0 (0%) | 0 (0%) | 0 (0%) |
| Others | 3 (9%) | 0 (0%) | 0 (0%) | 0 (0%) |

Hepatic

| | | | | |
|---|---|---|---|---|
| Abdominal pain | 1 (3%) | 0 (0%) | 0 (0%) | 0 (0%) |
| ALT increased | 7 (21%) | 2 (6%) | 0 (0%) | 0 (0%) |
| AST increased | 6 (18%) | 1 (3%) | 1 (3%) | 0 (0%) |
| GGT increased | 2 (6%) | 0 (0%) | 0 (0%) | 0 (0%) |
| Bilirubin raised | 7 (21%) | 0 (0%) | 0 (0%) | 0 (0%) |

Laboratory

| | | | | |
|---|---|---|---|---|
| Anemia | 5 (15%) | 0 (0%) | 0 (0%) | 0 (0%) |
| Leukopenia | 6 (18%) | 0 (0%) | 0 (0%) | 0 (0%) |
| Neutropenia | 1 (3%) | 0 (0%) | 0 (0%) | 0 (0%) |
| Thrombocytopenia | 4 (12%) | 0 (0%) | 0 (0%) | 0 (0%) |
| Hypothyroidism | 0 (0%) | 0 (0%) | 0 (0%) | 0 (0%) |
| Amylase increased | 0 (0%) | 0 (0%) | 0 (0%) | 0 (0%) |
| Lipase increased | 0 (0%) | 0 (0%) | 0 (0%) | 0 (0%) |
| Hyperglycemia | 1 (3%) | 0 (0%) | 0 (0%) | 0 (0%) |
| Hyponatremia | 4 (12%) | 0 (0%) | 0 (0%) | 0 (0%) |
| Hypokalemia | 6 (18%) | 0 (0%) | 0 (0%) | 0 (0%) |
| Hypercreatinine | 3 (9%) | 0 (0%) | 0 (0%) | 0 (0%) |
| Hypothyroidism | 5 (15%) | 0 (0%) | 0 (0%) | 0 (0%) |

Immune-related

| | | | | |
|---|---|---|---|---|
| Hepatitis | 2 (6%) | 3 (9%) | 0 (0%) | 0 (0%) |
| Dermatitis | 2 (6%) | 2 (6%) | 0 (0%) | 0 (0%) |
| Others immune- | 5 (15%) | 0 (0%) | 0 (0%) | 0 (0%) |
| related | | | | |

Abbreviations:
GI, gastrointestinal;
ALT: Alanine transaminase;
AST: Aspartate transaminase;
GGT: Gamma-glutamyl Transferase
*Data are number of patients with the worst toxicity grade

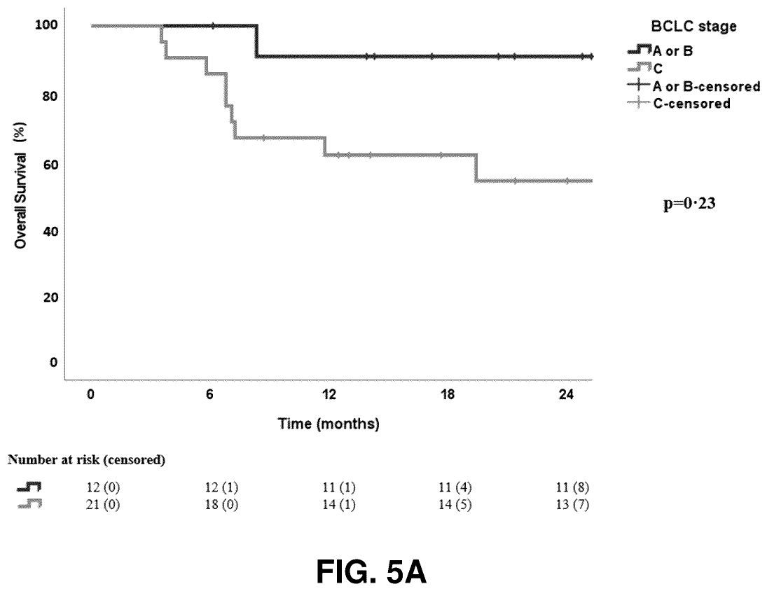

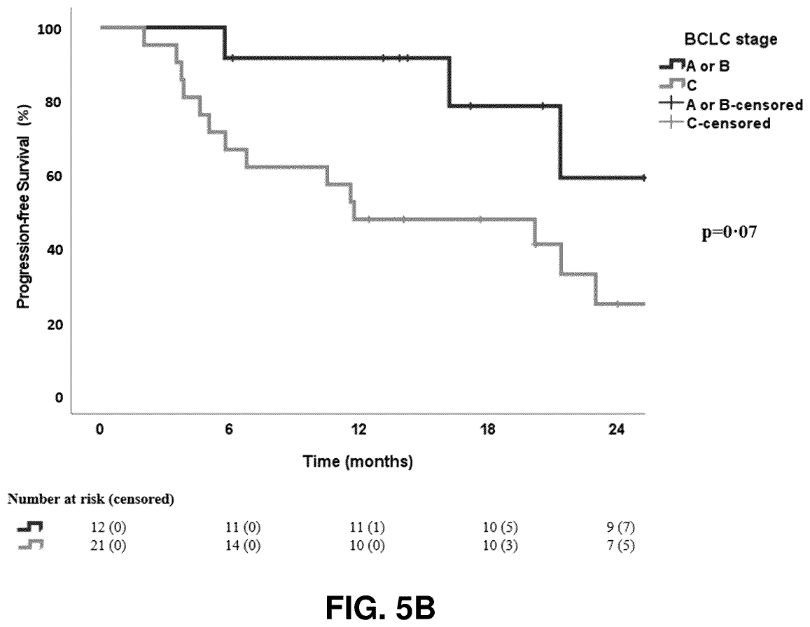

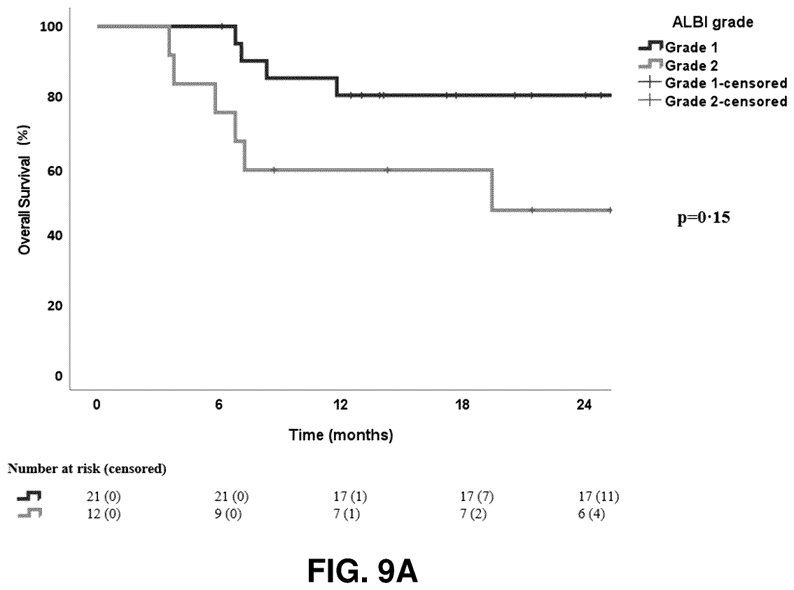

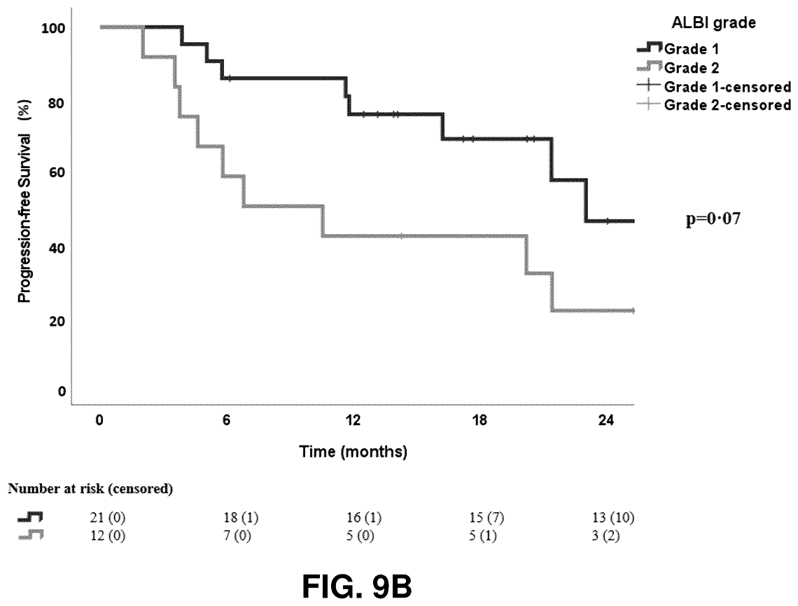

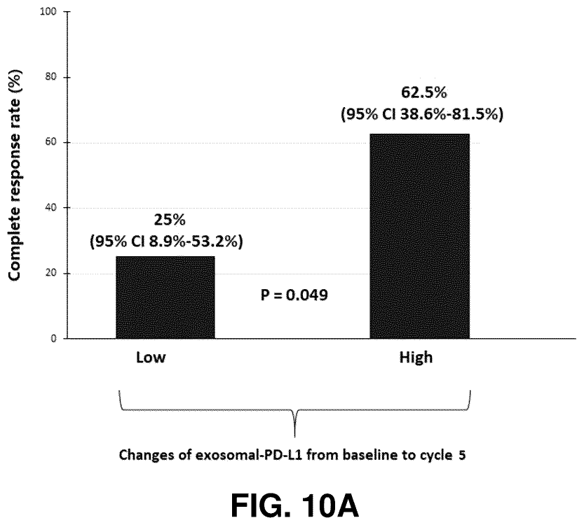

Although there were numerical differences in overall survival, objective response rate, and disease control rate between the two albumin-bilirubin grades, these differences were not statistically significant (Table 16; FIGS. 9A-9B). Dynamic changes in exosomal PD-L1 concentrations were evaluated in 24 patients. Patients who had a surge of exosomal PD-L1 at week 8 of avelumab (to 250 μg/mL or higher) had a better complete response rate than those who had exosomal PD-L1 concentrations lower than 250 μg/mL (10 [63%, 95% CI 39-82] of 16 patients with PD-L1≥250 pg/mL had a complete response vs 2 [25%, 95% CI 9-53] of 8 with PD-L1<250 pg/mL; p=0.049; FIGS. 10A-10B). The values are collected in Table 17. The median progression-free survival of patients with BCLC stage A and B beyond the up-to-7 criteria (n=12) was 26.7 months (95% CI 18.5-35.0) compared with 11.8 months (0-27.2) for patients (n=21) with BCLC stage C (p=0.070). The 24-month overall survival rate among patients without macrovascular invasion was 91% (82-100) compared with 57% (33-80) among patients with macrovascular invasion (p=0.23). Ten (83%) of 12 patients with BCLC stage A and B beyond the up-to-7 criteria became amenable to surgery compared with eight (38%) of 21 patients with BCLC stage C (p=0.012; Table 18; FIG. 5A-5B). Notably, more patients with albumin-bilirubin grade 2 had a Child-Pugh score progression of at least 2 points than did those with albumin-bilirubin grade 1 (Table 16).

TABLE 16
Comparison of clinical outcomes
between patients with Grade 1 ALBI vs. Grade 2 ALBI

| | Grade 1 ALBI | Grade 2 ALBI | P |
|---|---|---|---|
| | (n = 21) | (n = 12) | value |
| Overall survival | | | 0.15 |
| Median (months) | NR | 19.5 (3.5-30.4) | |
| 6-month rate, % (95% CI) | 100 (100-100) | 75 (41-91) | |
| 12-month rate, % (95% CI) | 80 (55-92) | 58 (27-80) | |
| 24-month rate, % (95% CI) | 80 (55-92) | 47 (17-72) | |
| Progression free survival | | | 0.07 |
| Median (months) | 22.2 (3.9-30.3) | 6.8 (2.0-28.1) | |
| 6-month rate, % (95% CI) | 86 (62-95) | 58 (27-80) | |
| 12-month rate, % (95% CI) | 76 (51-89) | 42 (15-67) | |
| 24-month rate, % (95% CI) | 46 (17-71) | 21 (4-48) | |
| ORR (CR + PR), No [symbol] % | 16 [symbol] 76 (53-92) | 6 [symbol] 50 (21-79) | 0.13 |
| (95% CD) | | | |
| CR [symbol] No [symbol] % (95% CI) | 11 [symbol] 52 (30-74) | 3 [symbol] 25 (6-57) | |
| PR [symbol] No [symbol] % (95% CD) | 5 [symbol] 24 (8-47) | 3 [symbol] 25 (6-57) | |
| SD [symbol] No [symbol] % (95% CI) | 1 [symbol] 5 (0-24) | 2 [symbol] 17 (2-48) | |
| PD [symbol] No [symbol] % (95% CI) | 4 [symbol] 19 (6-42) | 4 [symbol] 33 (10-65) | |
| DCR (CR + PR + SD [symbol] | 16 [symbol] 76 (53-92) | 6 [symbol] 50 (21-79) | 0.13 |
| months), No [symbol] % (95% CI) | | | |
| Amenable to curative surgery, | 14 (67) | 4 (33) | 0.06 |
| No [symbol] % | | | |
| Proportion of patients with CP | | | |
| score progression ≥2 (%) # | | | |
| 3 months | 0/18 (0) | 3/7 (43) | 0.003 |
| 6 months | 1/17 (6) | 2/5 (40) | 0.05 |
| 12 months | 0/13 (0) | 1/4 (25) | 0.06 |

Abbreviations:
ORR, objective response rate;
CR, complete response;
PR, partial response;
SD, stabledisease;
PD, progressive disease;
DCR, disease control rate;
DOR, duration of response;
PFS, progressionfree survival;
OS, overall survival;
NR, not reached
# CP score progression ≥2 refers to an increase in CP score by ≥2 at respective time point, in the absence of definite HCC progression. [symbol]
indicates data missing or illegible when filed

TABLE 17
Comparison of exosomal PD-L1 level among complete responders vs.
non-complete responders at baseline and at 1 month after immunotherapy

| Changes of exosomal PD-L1 | | Complete response rate, n, %, | |
|---|---|---|---|
| from baseline | N | 95% CI | P |
| Baseline to C5 (<250 pg/ml) | 8 | 2, 25 (9-53) | 0.049 |
| Baseline to C5 (≥250 pg/ml) | 16 | 10, 63 (39-82) | |
| Baseline exosomal PD-L1 | | | |
| level | | | |
| <15 pg/ml | 20 | 9, 45 (23-69) | 0.429 |
| ≥15 pg/ml | 10 | 3, 30 (7-66) | |

TABLE 18
Comparison of clinical outcomes between patients with BCLC A-B vs. BCLC C

| | BCLC A or B (n = 12) | BCLC C (n = 21) | P |
|---|---|---|---|
| | No macrovascular invasion | Macrovascular invasion | value |
| Overall survival | | | 0.23 |
| Median (months) | NR | NR | |
| 6-month rate, % (95% CI) | 100 (100-100) | 90 (77-100) | |
| 12-month rate, % (95% CI) | 91 (82-100) | 65 (43-86) | |
| 24-month rate, % (95% CI) | 91 (82-100) | 56 (33-80) | |
| Progression free survival | | | 0.07 |
| Median (months) | 26.7 (18.5-35.0) | 11.8 (0-27.2) | |
| 6-month rate, % (95% CI) | 92 (67-100) | 70 (50-90) | |
| 12-month rate, % (95% CI) | 92 (67-100) | 50 (28-72) | |
| 24-month rate, % (95% CI) | 59 (27-88) | 26 (3-49) | |
| ORR (CR+PR), No [symbol] % (95% CI) | 10 [symbol] 83 (52-98) | 12 [symbol] 60 (34-78) | 0.12 |
| CR [symbol] No [symbol] % (95% CI) | 6 [symbol] 50 (21-79) | 8 [symbol] 40 (18-69) | |
| PR [symbol] No [symbol] % (95% CI) | 4 [symbol] 33 (10-65) | 4 [symbol] 20 (6-42) | |
| SD [symbol] No [symbol] % (95% CI) | 1 [symbol] (0-38) | 1 [symbol] 5 (0-24) | |
| PD [symbol] No [symbol] % (95% CI) | 1 [symbol] 8 (0-38) | 7 [symbol] 35 (15-57) | |
| DCR (CR + PR + SD ≥ [symbol] | 11 [symbol] 92 (62-100) | 12 [symbol] 60 (36-81) | 0.58 |
| months), No [symbol] % (959% CI) | | | |
| Median DOR, No [symbol] months (range) | 21.0 (11.1-29.0) | 12.4 (0.2-26.73) | 0.38 |
| Amenable to curative surgery, | 10 (83) | 8 (38) | 0.01 |
| No [symbol] % | | | |
| Proportion of patients with CP | | | |
| score progression ≥ 2 (%) # | | | |
| 3 months | 1/11 (9) | 2/14 (14) | 0.69 |
| 6 months | 1/11 (9) | 2/11 (18) | 0.53 |
| 12 months | 0/8 (0) | 1/9 (11) | 0.33 |

Abbreviations:
ORR, objective response rate;
CR, complete response;
PR, partial response;
SD, stable disease;
PD, progressive disease;
DCR, disease control rate;
DOR, duration of response;
PFS, progression free survival;
OS, overall survival;
NR, not reached
# CP score progression ≥2 refers to an increase in CP score by ≥2 at respective time point, in the absence of definite HCC progression. [symbol]
indicates data missing or illegible when filed

In this trial, sequential TACE, stereotactic body radiotherapy, followed by PD-L1 blockade in patients with locally advanced unresectable hepatocellular carcinoma resulted in 55% of patients becoming amenable to curative treatment, and 12% of patients undergoing curative treatment. Moreover, 42% of patients who were enrolled in the trial had radiological complete response without surgery, and a 2-year overall survival rate of 92%. This is the first prospective clinical trial using sequential locoregional treatment combined with immunotherapy as conversion therapy for locally advanced unresectable hepatocellular carcinoma. The objective response rate of 67% is superior compared with that of standalone systemic therapy (14-40%) and TACE (30%) in similar patient populations.⁴⁻⁶ Over 60% of the cohort had BCLC stage C disease, representing a spectrum of hepatocellular carcinoma characterized by poor prognostic outcomes due to macrovascular invasion. According to current international guidelines, these patients are not candidates for locoregional therapies, and systemic therapy remains the standard of care.²¹

An individualized stereotactic body radiotherapy dose-allocation strategy was adopted to treat large, multifoxpatocellular carcinoma with macrovascular invasion.^{7,24} Patients enrolled in the STAR-FIT trial received radiation for all gross lesions; emerging evidence favored the comprehensive radiation of all tumor sites to improve immune access and reduce the immunosuppressive effects of bulky lesions.²⁵ The favorable local control and complete response rates observed in the STAR-FIT trial can be explained by the additive effect of TACE, radiotherapy, iodized oil staining to improve tumor target localisation,²⁶ and most importantly, the extra combined immunomodulatory effect of the combined regimen.^{8-12, 26} Preclinical data indicated that immune checkpoint inhibitors could sensitize the tumor to radiotherapy.²⁷ It was realized that immunotherapy can reduce the chance of out-of-field progression after locoregional therapy. Indeed, it was discovered that the immunomodulatory effect of TACE and stereotactic body radiotherapy can further augment the effect of immune checkpoint inhibitors in eradicating occult metastasis.

The safety profile of the triple therapy regimen was consistent with the safety profile previously observed with TACE, stereotactic body radiotherapy, or avelumab alone.⁶ All seven patients who developed a transient impairment of liver function that was grade 3 or worse due to TACE continued stereotactic body radiotherapy without delay. In terms of preventing Child-Pugh class deterioration, the START-FIT regimen compared favorably with repeated TACE.²⁹

In conclusion, the disclosed methods, as exemplified by the START-FIT regimen is useful as a conversion therapy for patients with locally advanced unresectable hepatocellular carcinoma. The disclosed methods represent a path for oncosurgical downstaging.

### REFERENCES

1. Sung H, Ferlay J, Siegel R L, et al. Global cancer statistics 2020: GLOBOCAN estimates of incidence and mortality worldwide for 36 cancers in 185 countries. CA Cancer J Clin 2021; 71:209-49.
2. Shim S J, Seong J, Han K H, Chon C Y, Suh C O, Lee J T. Local radiotherapy as a complement to incomplete transcatheter arterial chemoembolization in locally advanced hepatocellular carcinoma. Liver Int 2005; 25:1189-96.
3. Kudo M, Han K H, Ye S L, et al. A changing paradigm for the treatment of intermediate-stage hepatocellular carcinoma: Asia-Pacific primary liver cancer expert consensus statements. Liver Cancer 2020; 9:245-60.
4. Kudo M, Finn R S, Qin S, et al. Lenvatinib versus sorafenib in first-line treatment of patients with unresectable hepatocellular carcinoma: a randomised phase 3 non-inferiority trial. Lancet 2018; 391:1163-73.
5. Finn R S, Qin S, Ikeda M, et al. Atezolizumab plus bevacizumab in unresectable hepatocellular carcinoma. N Engl J Med 2020; 382:1894-905.
6. Finn R S, Ryoo B Y, Merle P, et al. Pembrolizumab as second-line therapy in patients with advanced hepatocellular carcinoma in KEYNOTE-240: a randomized, double-blind, phase iii trial. J Clin Oncol 2020; 38:193-202.
7. Bujold A, Massey C A, Kim J J, et al. Sequential phase I and II trials of stereotactic body radiotherapy for locally advanced hepatocellular carcinoma. J Clin Oncol 2013; 31:1631-39.
8. Yoon S M, Ryoo B Y, Lee S J, et al. Efficacy and safety of transarterial chemoembolization plus external beam radiotherapy vs sorafenib in hepatocellular carcinoma with macroscopic vascular invasion: a randomized clinical trial. JAMA Oncol 2018; 4:661-69.
9. Su T S, Lu H Z, Cheng T, et al. Long-term survival analysis in combined transarterial embolization and stereotactic body radiation therapy versus stereotactic body radiation monotherapy for unresectable hepatocellular carcinoma >5 cm. BMC Cancer 2016; 16:834.
10. Wong T C, Chiang C L, Lee A S, et al. Better survival after stereotactic body radiation therapy following transarterial chemoembolization in nonresectable hepatocellular carcinoma: a propensity score matched analysis. Surg Oncol 2019; 28: 228-35.
11. Sharabi A B, Lim M, DeWeese T L, Drake C G. Radiation and checkpoint blockade immunotherapy: radiosensitisation and potential mechanisms of synergy. Lancet Oncol 2015; 16: e498-509.
12. Greten T F, Mauda-Havakuk M, Heinrich B, Korangy F, Wood B J. Combined locoregional-immunotherapy for liver cancer. J Hepatol 2019; 70: 999-1007.
13. Sangro B, Gomez-Martin C, de la Mata M, et al. A clinical trial of CTLA-4 blockade with tremelimumab in patients with hepatocellular carcinoma and chronic hepatitis C. J Hepatol 2013; 59:81-88.
14. Chiang C L, Chan A C Y, Chiu K W H, Kong F S. Combined stereotactic body radiotherapy and checkpoint inhibition in unresectable hepatocellular carcinoma: a potential synergistic treatment strategy. Front Oncol 2019; 9:1157.
15. Chiang C L, Chiu K W H, Lee F A S, Kong F S, Chan A C Y. Combined stereotactic body radiotherapy and immunotherapy versus transarterial chemoembolization in locally advanced hepatocellular carcinoma: a propensity score matching analysis. Front Oncol 2021; 11:798832.
16. Ng K K C, Chok K S H, Chan A C Y, et al. Randomized clinical trial of hepatic resection versus radiofrequency ablation for early-stage hepatocellular carcinoma. Br J Surg 2017; 104:1775-84.
17. Yao F Y, Ferrell L, Bass N M, et al. Liver transplantation for hepatocellular carcinoma: expansion of the tumor size limits does not adversely impact survival. Hepatology 2001; 33:1394-403.
18. Velec M, Haddad C R, Craig T, et al. Predictors of liver toxicity following stereotactic body radiation therapy for hepatocellular carcinoma. Int J Radiat Oncol Biol Phys 2017; 97:939-46.
19. Simon R. Optimal two-stage designs for phase II clinical trials. Control Clin Trials 1989; 10:1-10.
20. Brookmeyer R, Crowley J. A confidence interval for the median survival time. Biometrics 1982; 38:29-41.
21. Bruix J, Chan S L, Galle P R, Rimassa L, Sangro B. Systemic treatment of hepatocellular carcinoma: An EASL position paper. J Hepatol 2021; 75:960-74.
22. Tai D, Loke K, Gogna A, et al. Radioembolisation with Y90-resin microspheres followed by nivolumab for advanced hepatocellular carcinoma (CA 209-678): a single arm, single centre, phase 2 trial. Lancet Gastroenterol Hepatol 2021; 6:1025-35.
23. Peng Z, Fan W, Zhu B, et al. Lenvatinib combined with transarterial chemoembolization as first-line treatment for advanced hepatocellular carcinoma: a phase III, randomized clinical trial (LAUNCH). J Clin Oncol 2022; published online August 3. https://doi. org/10.1200/JCO.22.00392.
24. Tse R V, Hawkins M, Lockwood G, et al. Phase I study of individualized stereotactic body radiotherapy for hepatocellular carcinoma and intrahepatic cholangiocarcinoma. J Clin Oncol 2008; 26:657-64.
25. Brooks E D, Chang J Y. Time to abandon single-site irradiation for inducing abscopal effects. Nat Rev Clin Oncol 2019; 16:123-35.
26. Chan M K, Lee V, Chiang C L, et al. Lipiodol versus diaphragm in 4D-CBCT-guided stereotactic radiotherapy of hepatocellular carcinomas. Strahlenther Onkol 2016; 192:92-101.
27. Vanpouille-Box C, Alard A, Aryankalayil M J, et al. DNA exonuclease Trex1 regulates radiotherapy-induced tumour immunogenicity. Nat Commun 2017; 8:15618.
28. Torok J A, Salama J K. Combining immunotherapy and radiotherapy for the STAR treatment. Nat Rev Clin Oncol 2019; 16:666-67.
29. Yasui Y, Tsuchiya K, Kurosaki M, et al. Up-to-seven criteria as a useful predictor for tumor downstaging to within Milan criteria and Child-Pugh grade deterioration after initial conventional transarterial chemoembolization. Hepatol Res 2018; 48:442-50.
30. Llovet J M, Lencioni R. mRECIST for HCC: performance and novel refinements. J Hepatol 2020; 72:288-306.

It is understood that the disclosed method and compositions are not limited to the particular methodology, protocols, and reagents described as these can vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “an,” and “the” include plural reference unless the context clearly dictates otherwise. Thus, for example, reference to “a treatment” includes a plurality of such treatments, reference to “the treatment” is a reference to one or more treatments and equivalents thereof known to those skilled in the art, and so forth.

Throughout the description and claims of this specification, the word “comprise” and variations of the word, such as “comprising” and “comprises,” means “including but not limited to,” and is not intended to exclude, for example, other additives, components, integers or steps.

“Optional” or “optionally” means that the subsequently described event, circumstance, or material may or may not occur or be present, and that the description includes instances where the event, circumstance, or material occurs or is present and instances where it does not occur or is not present.

Unless the context clearly indicates otherwise, use of the word “can” indicates an option or capability of the object or condition referred to. Generally, use of “can” in this way is meant to positively state the option or capability while also leaving open that the option or capability could be absent in other forms or embodiments of the object or condition referred to. Unless the context clearly indicates otherwise, use of the word “may” indicates an option or capability of the object or condition referred to. Generally, use of “may” in this way is meant to positively state the option or capability while also leaving open that the option or capability could be absent in other forms or embodiments of the object or condition referred to. Unless the context clearly indicates otherwise, use of “may” herein does not refer to an unknown or doubtful feature of an object or condition.

Ranges can be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e., a single number) can be selected as the quantity, value, or feature to which the range refers. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Although the description of materials, compositions, components, steps, techniques, etc. can include numerous options and alternatives, this should not be construed as, and is not an admission that, such options and alternatives are equivalent to each other or, in particular, are obvious alternatives.

Those skilled in the art will recognize or be able to ascertain using no more than routine experimentation many equivalents to the specific embodiments of the method and compositions described herein. Such equivalents are intended to be encompassed by the following claims.

We claim:

1. A method for treating a subject having hepatocellular carcinoma (HCC), the method comprising:
(a) treating the subject with transarterial chemoembolization (TACE);
(b) treating the subject with stereotactic body radiotherapy (SBRT); and
(c) treating the subject with an immune checkpoint inhibitor,
wherein the HCC comprises one or more tumors.

2. The method of claim 1, wherein the subject's HCC is locally advanced.

3. The method of claim 1, wherein the subject's HCC is unresectable.

4. The method of claim 1, wherein steps (a), (b), and (c) are performed sequentially in that order.

5. The method of claim 1, wherein the immune checkpoint inhibitor targets programmed cell death ligand 1 (PD-L1).

6. The method of claim 1, wherein the immune checkpoint inhibitor targets programmed cell death protein 1 (PD-1).

7. The method of claim 1, wherein the immune checkpoint inhibitor targets cytotoxic T-lymphocyte-associated protein 4 (CTLA-4).

8. The method of claim 1, wherein the immune checkpoint inhibitor targets lymphocyte-activation gene 3 (LAG-3).

9. The method of claim 1, wherein the immune checkpoint inhibitor comprises a therapeutic antibody.

10. The method of claim 9, wherein the therapeutic antibody is avelumab, atezolizumab, durvalumab, envafolimab, cosibelimab, or AUNP-12.

11. The method of claim 9, wherein the therapeutic antibody is pembrolizumab, nivolumab, cemiplimab, spartalizumab, camrelizumab, sintilimab, tislelizumab, toripalimab, dostarlimab, retifanlimab, AMP-224, or MEDI0680.

12. The method of claim 9, wherein the therapeutic antibody is ipilimumab or tremelimumab.

13. The method of claim 9, wherein the therapeutic antibody is relatlimab.

14. The method of claim 1, wherein TACE comprises infusing a chemotherapeutic agent and an embolic particle into an artery supplying blood to one or more tumors of the HCC.

15. The method of claim 14, wherein the chemotherapeutic agent and the embolic particle are infused together or separately.

16. The method of claim 14, wherein the chemotherapeutic agent and the embolic particle are infused sequentially.

17. The method of claim 14, wherein the embolic particle comprises a polyvinyl alcohol microsphere, superabsorbent microsphere, gelatin microsphere, or degradable starch microsphere.

18. The method of claim 1 further comprising:
resecting tumor tissue in the subject.

19. The method of claim 1 further comprising:
treating the subject with radiofrequency ablation.
